(19) United States
(12) Patent Application Publication
(10) Pub. No.: US 2014/0148498 A1
(43) Pub. Date: May 29, 2014

(54) METHODS AND COMPOSITIONS FOR THE TREATMENT OF MARFAN SYNDROME AND ASSOCIATED DISORDERS

(71) Applicants: Harry C. Dietz, Towson, MD (US); Jefferson J. Doyle, Baltimore, MD (US); Jennifer Pardo Habashi, Annapolis, MD (US); Tammy Holm, Olympia, WA (US)

(72) Inventors: Harry C. Dietz, Towson, MD (US); Jefferson J. Doyle, Baltimore, MD (US); Jennifer Pardo Habashi, Annapolis, MD (US); Tammy Holm, Olympia, WA (US)

(73) Assignee: THE JOHNS HOPKINS UNIVERSITY, Baltimore, MD (US)

(21) Appl. No.: 14/111,176

(22) PCT Filed: Apr. 13, 2012
(86) PCT No.: PCT/US12/33613

§ 371 Date: Jan. 15, 2014

Related U.S. Application Data
(60) Provisional application No. 61/475,491, filed on Apr. 14, 2011.

(51) Int. Cl. A61K 31/713; A61K 31/416; A61K 31/18

(52) U.S. Cl. CPC A61K 31/713; A61K 31/18; A61K 31/416
USPC 514/44 A; 514/601; 514/406; 536/24.5; 564/80; 548/358.5

(57) ABSTRACT

The instant invention provides methods and compositions for the treatment and prevention of Marfan syndrome and related diseases, disorders and conditions. The invention further provides pharmaceutical compositions and kits for the treatment and prevention of Marfan syndrome and related diseases, disorders and conditions.

### RELATED APPLICATIONS

This application claims the benefit of U.S. Provisional Application No. 61/475,491, filed Apr. 14, 2011 the entire contents of which is expressly incorporated herein by reference.

### GOVERNMENT SUPPORT

The following invention was supported at least in part by NIH Grant Nos. AR 4113-14, AR041135, and AR049698. Accordingly, the government has certain rights in the invention.

### BACKGROUND OF THE INVENTION

The Marfan syndrome (MFS) is a systemic disorder of connective tissue with autosomal dominant inheritance and a prevalence of approximately 1 per 5,000 population (Pyeritz, R. E. & McKusick, V. A. (1979) N Engl J Med. 300, 772-777). The syndrome shows no racial preference and both sexes are affected equally. It has been estimated that 25% of cases occur due to spontaneous mutations. While this condition shows high penetrance, marked interfamilial clinical variability is the rule (Pyeritz, R. E. et al. (1979) Birth Defects Orig Artic Ser. 15, 155-178). The lack of a specific biochemical or genetic marker of disease, coupled with the variability in clinical presentation, has frustrated diagnosis of equivocal cases and has likely contributed to a significant underestimation of the prevalence of disease.

The cardinal features of this disorder involve the ocular, skeletal, and cardiovascular systems. Cardiovascular pathology, including aortic root dilatation, dissection, and rupture, pulmonary artery dilatation, myxomatous valve changes with insufficiency of the mitral and aortic valves, and progressive myocardial dysfunction, is the leading cause of mortality in the MFS. The majority of fatal events associated with untreated MFS occur in early adult life. In a prospective study of 72 patients in 1972, the average age of death was 32 years (Murdoch, J. L. et al. (1972) N Engl J Med. 286, 804-808).

A recent reevaluation of life expectancy in the Marfan syndrome suggested that early diagnosis and refined medical and surgical management has greatly improved this situation (Silverman, D. I. et al. (1995) AmJ Cardiol. 75, 157-160). Nevertheless, MFS continues to be associated with significant morbidity and selected subgroups are refractory to therapy and continue to show early mortality Morse, R. P. et al. (1990) Pediatrics. 86, 888-895; Sisk, H. E., et al. (1983) Am J Cardiol. 52, 353-358). In a review of 54 patients diagnosed during infancy, Morse et al. reported that 89% had serious cardiac pathology, and that cardiac disease was progressive despite standard care (22% died during childhood, 16% before age 1 year). In the more classic form of Marfan syndrome it is estimated that greater than 90% of individuals will have a cardiovascular ‘event’ during their lifetime, defined as the need for prophylactic surgical repair of the aortic root or death due to aortic dissection (Gillinov, A. M., et al. (1997) Ann Thorac Surg. 64, 1140-1144; discussion 1144-1145; Pyeritz, R. E. (1993) Semin Thorac Cardiovasc Surg. 5, 11-16; Silverman, D. I., et al. (1995) J Am Coll Cardiol. 26, 1062-1067; Gott, V. L., et al. (1999) N Engl J Med. 340, 1307-1313). Ocular and skeletal morbidity is less easily quantified (Maumenee, I. H. et al. (1981) Trans Am Ophthalmol Soc. 79, 684-733; Magid, D., et al. (1990) AJR Am J Roentgenol. 155, 99-104; Sponseller, P. D., et al. (1995) J Bone Joint Surg Am. 77, 867-876). Approximately 60% of individuals with MFS have lens dislocation, often requiring surgical aphakia for optimal management. Retinal detachment and glaucoma can cause devastating visual impairment.

Skeletal involvement is evident in nearly all people with MFS. Progressive anterior chest deformity or scoliosis can cause cardiopulmonary dysfunction and commonly require surgical correction. Joint instability can cause physical disability and predispose to premature arthritis. Lung disease most commonly manifests with spontaneous pneumothorax and has been identified in 4-11% of MFS patients (Wood, J. R., et al. (1984) Thorax. 39, 780-784; Hall, J. R., et al. (1984) Ann Thorac Surg. 37, 500-504). Pathologic findings include upper lobe bullae with or without diffuse fixed obstructive airway disease that can be progressive and has traditionally been equated with destructive emphysema (Lipton, R. A., et al. (1971) Am Rev Respir Dis. 104, 924; Dominguez, R., et al. (1987) Pediatr Radiol. 17, 365-369) The majority of patients with MFS display a marked deficiency in skeletal muscle mass and fat stores despite adequate caloric intake and no evidence for malabsorption (Behan, W. M., et al. (2003) J Neurol Neurosurg Psychiatry. 74, 633-638; H. H., et al. (1973) Neurology. 23, 1257-1268; Gross, M. L., et al. (1980) J Neurol Sci. 46, 105-112; Joyce, D. A., et al. (1984) Aust N Z J Med. 14, 495-499). Evidence for skeletal muscle myopathy, including decreased strength and tone, has been observed in a subset of affected individuals and may contribute to decreased functional performance, respiratory insufficiency, ocular misalignment, and altered development of the skeleton including kyphosis and scoliosis.

An increasing challenge is to define the “new” natural history of MFS now that many individuals are surviving their predisposition for early aortic root dissection; already appreciated aging-associated phenotypes include a predisposition for dissection of the descending thoracic and abdominal aorta. Thus, despite advances in our ability to increase the length of life for many individuals with MFS, there is ample opportunity to improve the quality of life for the majority of affected individuals.

In 1991 a traditional positional-candidate analysis culminated with the demonstration of disease producing mutations in the FBN1 gene on chromosome 15q21.1 that encodes fibrillin-1 (Dietz, H. C., et al. (1991) Nature. 352, 337-339). Since that time, there has been generation and characterization of multiple mouse models of Marfan syndrome. This work has truly revolutionized the understanding of the pathogenesis of disease and has lead to exciting strategies for the treatment of the multisystem pathogenesis of Marfan syndrome.

Many of the features of Marfan syndrome are common in the general population and represent a tremendous public health burden. These include aortic aneurysm (1-2% of the population at large), mitral valve prolapse (˜7%), emphysema (11%), scoliosis (0.5%), cataract (30%), arthritis (very common), and myopathy (many common genetic and acquired forms).

Accordingly, a need exists for methods and compositions for the treatment of Marfan syndrome and associated diseases, disorders and conditions, e.g., diseases, disorders and conditions associated with aberrant TGF-β expression.

### SUMMARY OF THE INVENTION

As described below, the present disclosure features compositions and methods for the treatment of Marfan syndrome diseases and disorders.

In one aspect, the present disclosure generally features a method of treating a patient having or at risk of developing a disease or disorder characterized by aberrant TGFβ expression or activity the method involving administering to the subject an effective amount of an agent that modulates the activity of noncanonical TGFβ signaling; thereby treating the patient.

In another aspect, the disclosure features a method of treating a patient having Marfan syndrome or a Marfan-associated condition the method involving administering to the subject an effective amount of an agent that modulates the activity of noncanonical TGFβ signaling; thereby treating the patient.

In yet another aspect, the disclosure features a method of treating a patient having Marfan syndrome or a Marfan-associated condition the method involving administering to the subject an effective amount of an agent that selectively activates Angiotensin II Receptor Type 2 (AT2); thereby treating the patient.

In a further aspect, the disclosure features a method of treating a patient having or at risk of developing a disease or disorder caused by mutation in the fibrillin 1 gene (FBN1) the method comprising administering to the subject an effective amount of an agent that modulates the activity of noncanonical TGFβ signaling; thereby treating the patient.

In additional aspects, the disclosure features a pharmaceutical composition for the treatment of a disease or disorder characterized by aberrant TGFβ expression or activity where the pharmaceutical composition contains an agent that modulates the activity of noncanonical TGFβ signaling.

In yet additional aspects, the disclosure features a kit for the treatment of a disease or disorder characterized by aberrant TGFβ expression or activity where the kit contains a pharmaceutical composition that contains an agent that modulates the activity of noncanonical TGFβ signaling and instructions for use.

In further aspects, the disclosure features a method of optimizing the dosing regimen or route of delivery for a Marfan syndrome therapeutic the method involving a) measuring noncanonical TGFβ signaling status in a sample from a patient; b) increasing the dosage or altering the route of delivery of the Marfan syndrome therapeutic administered to the subject if the noncanonical TGFβ signaling is above a threshold amount; and c) repeating steps a) and b) until the noncanoncial TGFβ signaling is below a threshold amount.

In various embodiments of any of the above aspects or any other aspect of the disclosure delineated herein, the disease or disorder is Marfan syndrome or a clinical condition associated with Marfan syndrome. In another embodiment the disease or disorder is an aneurysm, an aortic aneurysm, or emphysema. In yet another embodiment the disease or disorder is an aneurysm. In further embodiments, the disease or disorder is a lung disease or disorder. In yet additional embodiments, the lung disease or disorder is selected from the group consisting of emphysema, pneumothorax, and COPD. In additional embodiments, the agent is a noncanonical TGFβ signaling pathway inhibitor. In an another embodiment, the agent is an inhibitor of a molecule whose activity is required for ERK1/2 activation. In a further embodiment the agent is an inhibitor of MEK, ERK1/2, or JNK1. In yet another embodiment, the agent is an inhibitor of ERK1/2. In an additional embodiment, the agent is selected from the group consisting of SP600125, U0126, and RDEA119. In further embodiments, the agent is a siRNA or shRNA specific for a regulator of the noncanonical TGFβ signaling pathway. In yet another embodiment, the siRNA or shRNA is specific for the nucleic acid molecule set forth as SEQ ID NO:1, SEQ ID NO:2, SEQ ID NO:3, SEQ ID NO:4, or SEQ ID NO:5. In other embodiments, the agent is a selective agonist of AT2. In yet other embodiments, the agonist is selected from the group consisting of a small molecule, a polypeptide, an aptamer, and an antibody or antigen-binding fragment thereof. In additional embodiments, the disease or disorder is tissue fibrosis or scleroderma. In yet further embodiments, the noncanonical TGFβ signaling status is MEK activity, ERK1/2 activity or JNK1 activity.

### DEFINITIONS

By “noncanonical TGFβ signaling” is meant any non-Smad mediated signaling in response to TGFβ. A non-limiting illustrative example of noncanonical TGFβ signaling is TGFβ mediated signaling through the ERK1/2 pathway.

By “Extracellular signal-regulated kinase 1 and 2” or “ERK1/2” is meant a polypeptide having the amino acid sequence defined by accession numbers P28482.3 and P27361.4. An illustrative amino acid sequence (SEQ ID NO:6) of ERK2 is:

```
    1 maaaaaagag pemvrgqvfd vgprytnlsy igegaygmvc saydnvnkvr vaikkispfe
  61 hqtycqrtlr eikillrfrh eniigindii raptieqmkd vyivqdlmet dlykllktqh
121 lsndhicyfl yqilrglkyi hsanvlhrdl kpsnlllntt cdlkicdfgl arvadpdhdh
181 tgflteyvat rwyrapeiml nskgytksid iwsvgcilae mlsnrpifpg khyldqlnhi
241 lgilgspsqe dlnciinlka rnyllslphk nkvpwnrlfp nadskaldll dkmltfnphk
301 rieveqalah pyleqyydps depiaeapfk fdmelddlpk eklkelifee tarfqpgyrs
```

The corresponding nucleic acid sequence (SEQ ID NO:1) encoding ERK2 is:

```
      1 acataatttc tggagccctg taccaacgtg tggccacata ttctgtcagg aaccctgtgt
    61 gatcatggtc tggatctgca acacgggcca ggccaaagtc acagatcttg agatcacagg
  121 tggtgttgag cagcaggcag gcaggcaatc ggtccgagtg gctgtcggct cttcagctct
  181 ccgctcggcg tcttccttcc tctcccggtc agcgtcggcg gctgcaccgg cggcgggcag
  241 tcctgcggga ggggcgacaa gagctgaggc gcggccgccg agcgtcgagc tcagcgcggc
  301 ggaggcggcg gcggcccggc agccaacatg gcggcggcgg cggcggcggg cgcgggcccg
  361 gagatggtcc gcgggcaggt gttcgacgtg gggccgcgct acaccaacct ctcgtacatc
  421 ggcgagggcg cctacggcat ggtgtgctct gcttatgata atgtcaacaa agttcgagta
  481 gctatcaaga aaatcagccc ctttgagcac cagacctact gccagagaac cctgagggag
  541 ataaaaatct tactgcgctt cagacatgag aacatcattg gaatcaatga cattattcga
  601 gcaccaacca tcgagcaaat gaaagatgta tatatagtac aggacctcat ggaaacagat
  661 ctttacaagc tcttgaagac acaacacctc agcaatgacc atatctgcta ttttctctac
  721 cagatcctca gagggttaaa atatatccat tcagctaacg ttctgcaccg tgacctcaag
  781 ccttccaacc tgctgctcaa caccacctgt gatctcaaga tctgtgactt tggcctggcc
  841 cgtgttgcag atccagacca tgatcacaca gggttcctga cagaatatgt ggccacacgt
  901 tggtacaggg ctccagaaat tatgttgaat tccaagggct acaccaagtc cattgatatt
  961 tggtctgtag gctgcattct ggcagaaatg ctttccaaca ggcccatctt tccagggaag
1021 cattatcttg accagctgaa tcacattttg ggtattcttg gatccccatc acaagaagac
1081 ctgaattgta taataaattt aaaagctagg aactatttgc tttctcttcc acacaaaaat
1141 aaggtgccat ggaacaggct gttcccaaat gctgactcca aagctctgga cttattggac
1201 aaaatgttga cattcaaccc acacaagagg attgaagtag aacaggctct ggcccaccca
1261 tatctggagc agtattacga cccgagtgac gagcccatcg ccgaagcacc attcaagttc
1321 gacatggaat tggatgactt gcctaaggaa aagctaaaag aactaatttt tgaagagact
1381 gctagattcc agccaggata cagatcttaa atttgtcagg acaagggctc agaggactgg
1441 acgtgctcag acatcggtgt tcttcttccc agttcttgac ccctggtcct gtctccagcc
1501 cgtcttggct tatccacttt gactcctttg agccgtttgg aggggcggtt tctggtagtt
1561 gtggctttta tgctttcaaa gaatttcttc agtccagaga attcactggc c
```

An illustrative sequence (SEQ ID NO:7) of ERK1 is:

```
    1 maaaaaqggg ggeprrtegv gpgvpgevem vkgqpfdvgp rytqlqyige gaygmvssay
  61 dhvrktrvai kkispfehqt ycqrtlreiq illrfrhenv igirdilras tleamrdvyi
121 vqdlmetdly kllksqqlsn dhicyflyqi lrglkyihsa nvlhrdlkps nllinttcdl
181 kicdfglari adpehdhtgf lteyvatrwy rapeimlnsk gytksidiws vgcilaemls
241 nrpifpgkhy ldqlnhilgi lgspsqedln ciinmkarny lqslpsktkv awaklfpksd
301 skaldlldrm ltfnpnkrit veealahpyl eqyydptdep vaeepftfam elddlpkerl
361 kelifcreta rfcrpavlea p
```

The corresponding nucleic acid sequence (SEQ ID NO:2) encoding ERK1 is:

```
      1 cgttcctcgg cgccgccggg gccccagagg gcagcggcag caacagcagc agcagcagca
    61 gcgggagtgg agatggcggc ggcggcggct caggggggcg ggggcgggga gccccgtaga
  121 accgaggggg tcggcccggg ggtcccgggg gaggtggaga tggtgaaggg gcagccgttc
  181 gacgtgggcc cgcgctacac gcagttgcag tacatcggcg agggcgcgta cggcatggtc
  241 agctcggcct atgaccacgt gcgcaagact cgcgtggcca tcaagaagat cagccccttc
  301 gaacatcaga cctactgcca gcgcacgctc cgggagatcc agatcctgct gcgcttccgc
  361 catgagaatg tcatcggcat ccgagacatt ctgcgggcgt ccaccctgga agccatgaga
  421 gatgtctaca ttgtgcagga cctgatggag actgacctgt acaagttgct gaaaagccag
  481 cagctgagca atgaccatat ctgctacttc ctctaccaga tcctgcgggg cctcaagtac
  541 atccactccg ccaacgtgct ccaccgagat ctaaagccct ccaacctgct cagcaacacc
  601 acctgcgacc ttaagatttg tgatttcggc ctggcccgga ttgccgatcc tgagcatgac
  661 cacaccggct tcctgacgga gtatgtggct acgcgctggt accgggcccc agagatcatg
  721 ctgaactcca agggctatac caagtccatc gacatctggt ctgtgggctg cattctggct
  781 gagatgctct ctaaccggcc catcttccct ggcaagcact acctggatca gctcaaccac
  841 attctgggca tcctgggctc cccatcccag gaggacctga attgtatcat caacatgaag
  901 gcccgaaact acctacagtc tctgccctcc aagaccaagg tggcttgggc caagcttttc
  961 cccaagtcag actccaaagc ccttgacctg ctggaccgga tgttaacctt taaccccaat
1021 aaacggatca cagtggagga agcgctggct cacccctacc tggagcagta ctatgacccg
1081 acggatgagc cagtggccga ggagcccttc accttcgcca tggagctgga tgacctacct
1141 aaggagcggc tgaaggagct catcttccag gagacagcac gcttccagcc cggagtgctg
1201 gaggccccct agcccagaca gacatctctg caccctgggg cctggacctg cctcctgcct
1261 gcccctctcc cgccagactg ttagaaaatg gacactgtgc ccagcccgga ccttggcagc
1321 ccaggccggg gtggagcatg ggcctggcca cctctctcct ttgctgaggc ctccagcttc
1381 aggcaggcca aggccttctc ctccccaccc gccctcccca cggggcctcg ggagctcagg
1441 tggccccagt tcaatctccc gctgctgctg ctgctgcgcc cttaccttcc ccagcgtccc
1501 agtctctggc agttctggaa tggaagggtt ctggctgccc caacctgctg aagggcagag
1561 gtggagggtg gggggcgctg agtagggact cagggccatg cctgcccccc tcatctcatt
1621 caaaccccac cctagtttcc ctgaaggaac attccttagt ctcaagggct agcatccctg
1681 aggagccagg ccgggccgaa tcccctccct gtcaaagctg tcacttcgcg tgccctcgct
1741 gcttctgtgt gtggtgagca gaagtggagc tggggggcgt ggagagcccg gcgcccctgc
1801 cacctccctg acccgtctaa tatataaata tagagatgtg tctatggctg aaaaaaaaaa
1861 aaaaaa
```

By “c-Jun N-terminal Kinase 1” or “JNK1” is meant a polypeptide having the amino acid sequence of accession number P45983. An illustrative amino acid sequence (SEQ ID NO:8) of JNK1 is:

```
    1 msrskrdnnf ysveigdstf tvlkryqnlk pigsgaggiv caaydailer nvaikklsrp
  61 fqnqthakra yrelvlmkcv nhkniiglln vftpqkslee fqdvyivmel mdanlcqviq
121 meldhermsy llyqmlcgik hlhsagiihr dlkpsnivvk sdctlkildf glartagtsf.
181 mmtpyvvtry yrapevilgm gykenvdlws vgcimgemvc hkilfpgrdy idqwnkvieq
241 lgtpcpefmk klqptvrtyv enrpkyagys feklfpdvlf padsehnklk asqardllsk
301 mlvidaskri svdealqhpy invwydpsea eapppkipdk qlderehtie ewkeliykev
361 mdleertkng virgqpsplg aavingsqhp sssssvndvs smstdptlas dtdssleaaa
421 gplgccr
```

The corresponding nucleic acid sequence (SEQ ID NO:3) encoding JNK1 is:

```
      1 cattaattgc ttgccatcat gagcagaagc aagcgtgaca acaattttta tagtgtagag
    61 attggagatt ctacattcac agtcctgaaa cgatatcaga atttaaaacc tataggctca
  121 ggagctcaag gaatagtatg cgcagcttat gatgccattc ttgaaagaaa tgttgcaatc
  181 aagaagctaa gccgaccatt tcagaatcag actcatgcca agcgggccta cagagagcta
  241 gttcttatga aatgtgttaa tcacaaaaat ataattggcc ttttgaatgt tttcacacca
  301 cagaaatccc tagaagaatt tcaagatgtt tacatagtca tggagctcat ggatgcaaat
  361 ctttgccaag tgattcagat ggagctagat catgaaagaa tgtcctacct tctctatcag
  421 atgctgtgtg gaatcaagca ccttcattct gctggaatta ttcatcggga cttaaagccc
  481 agtaatatag tagtaaaatc tgattgcact ttgaagattc ttgacttcgg tctggccagg
  541 actgcaggaa cgagttttat gatgacgcct tatgtagtga ctcgctacta cagagcaccc
  601 gaggtcatcc ttggcatggg ctacaaggaa aacgtggatt tatggtctgt ggggtgcatt
  661 atgggagaaa tggtttgcca caaaatcctc tttccaggaa gggactatat tgatcagtgg
  721 aataaagtta ttgaacagct tggaacacca tgtcctgaat tcatgaagaa actgcaacca
  781 acagtaagga cttacgttga aaacagacct aaatatgctg gatatagctt tgagaaactc
  841 ttccctgatg tccttttccc agctgactca gaacacaaca aacttaaagc cagtcaggca
  901 agggatttgt tatccaaaat gctggtaata gatgcatcta aaaggatctc tgtagatgaa
  961 gctctccaac acccgtacat caatgtctgg tatgatcctt ctgaagcaga agctccacca
1021 ccaaagatcc ctgacaagca gttagatgaa agggaacaca caatagaaga gtggaaagaa
1081 ttgatatata aggaagttat ggacttggag gagagaacca agaatggagt tatacggggg
1141 cagccctctc ctttagcaca ggtgcagcag tgatcaatgg ctctcagcat ccatcatcat
1201 cgtcgtctgt caatgatgtg tcttcaatgt caacagatcc gactttggcc tctgatacag
1261 acagcagtct agaagcagca gctgggcctc tgggctgctg tagatgacta cttgggccat
1321 cggggggtgg gagggatggg gagtcggtta gtcattgata gaactacttt gaaaacaatt
1381 cagtggtctt atttttgggt gatttttcaa aaaatgta
```

By “MEK” also referred to as “dual specificity mitogen-activated protein kinase kinase” is meant a polypeptide having the amino acid sequence defined by accession numbers NP_{—}002746.1 and NP_{—}109587.1. An illustrative amino acid sequence (SEQ ID NO: 9) of MEK1 is:

```
    1 mpkkkptpiq lnpapdgsav ngtssaetnl ealqkkleel eldeqqrkrl eafltqkqkv
  61 gelkdddfek iselgagngg vvfkvshkps glvmarklih leikpairnq iirelqvlhe
121 cnspyivgfy gafysdgeis icmehmdggs ldqvlkkagr ipeqilgkvs iavikgltyl
181 rekhkimhrd vkpsnilvns rgeiklcdfg vsgqlidsma nsfvgtrsym sperlqgthy
241 svqsdiwsmg lslvemavgr ypipppdake lelmfgcqve gdaaetpprp rtpgrplssy
301 gmdsrppmai felldyivne pppklpsgvf slefqdfvnk cliknpaera dlkqlmvhaf
361 ikrsdaeevd fagwlcstig lnqpstptha agv
```

The corresponding nucleic acid sequence (SEQ ID NO:4) encoding MEK1 is:

```
      1 aggcgaggct tccccttccc cgcccctccc ccggcctcca gtccctccca gggccgcttc
    61 gcagagcggc taggagcacg gcggcggcgg cactttcccc ggcaggagct ggagctgggc
  121 tctggtgcgc gcgcggctgt gccgcccgag ccggagggac tggttggttg agagagagag
  181 aggaagggaa tcccgggctg ccgaaccgca cgttcagccc gctccgctcc tgcagggcag
  241 cctttcggct ctctgcgcgc gaagccgagt cccgggcggg tggggcgggg gtccactgag
  301 accgctaccg gcccctcggc gctgacggga ccgcgcgggg cgcacccgct gaaggcagcc
  361 ccggggcccg cggcccggac ttggtcctgc gcagcgggcg cggggcagcg cagcgggagg
  421 aagcgagagg tgctgccctc cccccggagt tggaagcgcg ttacccgggt ccaaaatgcc
  481 caagaagaag ccgacgccca tccagctgaa cccggccccc gacggctctg cagttaacgg
  541 gaccagctct gcggagacca acttggaggc cttgcagaag aagctggagg agctagagct
  601 tgatgagcag cagcgaaagc gccttgaggc ctttcttacc cagaagcaga aggtgggaga
  661 actgaaggat gacgactttg agaagatcag tgagctgggg gctggcaatg gcggtgtggt
  721 gttcaaggtc tcccacaagc cttctggcct ggtcatggcc agaaagctaa ttcatctgga
  781 gatcaaaccc gcaatccgga accagatcat aagggagctg caggttctgc atgagtgcaa
  841 ctctccgtac atcgtgggct tctatggtgc gttctacagc gatggcgaga tcagtatctg
  901 catggagcac atggatggag gttctctgga tcaagtcctg aagaaagctg gaagaattcc
  961 tgaacaaatt ttaggaaaag ttagcattgc tgtaataaaa ggcctgacat atctgaggga
1021 gaagcacaag atcatgcaca gagatgtcaa gccctccaac atcctagtca actcccgtgg
1081 ggagatcaag ctctgtgact ttggggtcag cgggcagctc atcgactcca tggccaactc
1141 cttcgtgggc acaaggtcct acatgtcgcc agaaagactc caggggactc attactctgt
1201 gcagtcagac atctggagca tgggactgtc tctggtagag atggcggttg ggaggtatcc
1261 catccctcct ccagatgcca aggagctgga gctgatgttt gggtgccagg tggaaggaga
1321 tgcggctgag accccaccca ggccaaggac ccccgggagg ccccttagct catacggaat
1381 ggacagccga cctcccatgg caatttttga gttgttggat tacatagtca acgagcctcc
1441 tccaaaactg cccagtggag tgttcagtct ggaatttcaa gattttgtga ataaatgctt
1501 aataaaaaac cccgcagaga gagcagattt gaagcaactc atggttcatg cttttatcaa
1561 gagatctgat gctgaggaag tggattttgc aggttggctc tgctccacca tcggccttaa
1621 ccagcccagc acaccaaccc atgctgctgg cgtctaagtg tttgggaagc aacaaagagc
1681 gagtcccctg cccggtggtt tgccatgtcg cttttgggcc tccttcccat gcctgtctct
1741 gttcagatgt gcatttcacc tgtgacaaag gatgaagaac acagcatgtg ccaagattct
1801 actcttgtca tttttaatat tactgtcttt attcttatta ctattattgt tcccctaagt
1861 ggattggctt tgtgcttggg gctatttgtg tgtatgctga tgatcaaaac ctgtgccagg
1921 ctgaattaca gtgaaatttt ggtgaatgtg ggtagtcatt cttacaattg cactgctgtt
1981 cctgctccat gactggctgt ctgcctgtat tttcgggatt ctttgacatt tggtggtact
2041 ttattcttgc tgggcatact ttctctctag gagggagcct tgtgagatcc ttcacaggca
2101 gtgcatgtga agcatgcttt gctgctatga aaatgagcat cagagagtgt acatcatgtt
2161 attttattat tattatttgc ttttcatgta gaactcagca gttgacatcc aaatctagcc
2221 agagcccttc actgccatga tagctggggc ttcaccagtc tgtctactgt ggtgatctgt
2281 agacttctgg ttgtatttct atatttattt tcagtatact gtgtgggata cttagtggta
2341 tgtctcttta agttttgatt aatgtttctt aaatggaatt attttgaatg tcacaaattg
2401 atcaagatat taaaatgtcg gatttatctt tccccatatc caagtaccaa tgctgttgta
2461 aacaacgtgt atagtgccta aaattgtatg aaaatccttt taaccatttt aacctagatg
2521 tttaacaaat ctaatctctt attctaataa atatactatg aaataaaaaa aaaaggatga
2581 aagctaaaaa aaaaaaaaaa aaa
```

An illustrative amino acid sequence (SEQ ID NO:10) of MEK2 is:

```
    1 mlarrkpvlp altinptiae gpsptsegas eanlvdlqkk leeleldeqq kkrleafltq
  61 kakvgelkdd dferiselga gnggvvtkvq hrpsglimar klihleikpa irnqiirelq
121 vlhecnspyi vgfygafysd geisicmehm dggsldqvlk eakripeeil gkvsiavlrg
181 laylrekhqi mhrdvkpsni lvnsrgeikl cdfgvsgqli dsmansfvgt rsymaperlq
241 gthysvqsdi wsmglslvel avgrypippp dakeleaifg rpvvdgeege phsisprprp
301 pgrpvsghgm dsrpamaife lldyivnepp pklpngvftp dfqefvnkcl iknpaeradl
361 kmltnhtfik rseveevdfa gwlcktlrin qpgtptrtav
```

The corresponding nucleic acid sequence (SEQ ID NO:5) encoding MEK2 is:

```
      1 cccctgcctc tcggactcgg gctgcggcgt cagccttctt cgggcctcgg cagcggtagc
    61 ggctcgctcg cctcagcccc agcgcccctc ggctaccctc ggcccaggcc cgcagcgccg
  121 cccgccctcg gccgccccga cgccggcctg ggccgcggcc gcagccccgg gctcgcgtag
  181 gcgccgaccg ctcccggccc gccccctatg ggccccggct agaggcgccg ccgccgccgg
  241 cccgcggagc cccgatgctg gcccggagga agccggtgct gccggcgctc accatcaacc
  301 ctaccatcgc cgagggccca tcccctacca gcgagggcgc ctccgaggca aacctggtgg
  361 acctgcagaa gaagctggag gagctggaac ttgacgagca gcagaagaag cggctggaag
  421 cctttctcac ccagaaagcc aaggtcggcg aactcaaaga cgatgacttc gaaaggatct
  481 cagagctggg cgcgggcaac ggcggggtgg tcaccaaagt ccagcacaga ccctcgggcc
  541 tcatcatggc caggaagctg atccaccttg agatcaagcc ggccatccgg aaccagatca
  601 tccgcgagct gcaggtcctg cacgaatgca actcgccgta catcgtgggc ttctacgggg
  661 ccttctacag tgacggggag atcagcattt gcatggaaca catggacggc ggctccctgg
  721 accaggtgct gaaagaggcc aagaggattc ccgaggagat cctggggaaa gtcagcatcg
  781 cggttctccg gggcttggcg tacctccgag agaagcacca gatcatgcac cgagatgtga
  841 agccctccaa catcctcgtg aactctagag gggagatcaa gctgtgtgac ttcggggtga
  901 gcggccagct catcgactcc atggccaact ccttcgtggg cacgcgctcc tacatggctc
  961 cggagcggtt gcagggcaca cattactcgg tgcagtcgga catctggagc atgggcctgt
1021 ccctggtgga gctggccgtc ggaaggtacc ccatcccccc gcccgacgcc aaagagctgg
1081 aggccatctt tggccggccc gtggtcgacg gggaagaagg agagcctcac agcatctcgc
1141 ctcggccgag gccccccggg cgccccgtca gcggtcacgg gatggatagc cggcctgcca
1201 tggccatctt tgaactcctg gactatattg tgaacgagcc acctcctaag ctgcccaacg
1261 gtgtgttcac ccccgacttc caggagtttg tcaataaatg cctcatcaag aacccagcgg
1321 agcgggcgga cctgaagatg ctcacaaacc acaccttcat caagcggtcc gaggtggaag
1381 aagtggattt tgccggctgg ttgtgtaaaa ccctgcggct gaaccagccc ggcacaccca
1441 cgcgcaccgc cgtgtgacag tggccgggct ccctgcgtcc cgctggtgac ctgcccaccg
1501 tccctgtcca tgccccgccc ttccagctga ggacaggctg gcgcctccac ccaccctcct
1561 gcctcacccc tgcggagagc accgtggcgg ggcgacagcg catgcaggaa cgggggtctc
1621 ctctcctgcc cgtcctggcc ggggtgcctc tggggacggg cgacgctgct gtgtgtggtc
1681 tcagaggctc tgcttcctta ggttacaaaa caaaacaggg agagaaaaag caaaaaaaaa
1741 aaaaaaaaaa aaaaaaaaa
```

By “Noncanonical TGFβ signaling inhibitor” is meant any agent that inhibits noncanonical TGFβ signaling.

By “RDEA119” is meant a selective inhibitor of mitogen activated ERK kinase (MEK) that has the following structure:

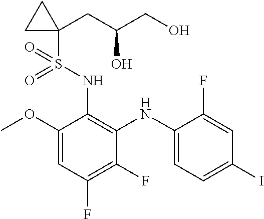 [SMILES: COc1cc(F)c(F)c(Nc2ccc(I)cc2F)c1NS(=O)(=O)C1(C[C@H](O)CO)CC1]

By “SP600125” is meant a small molecule inhibitor of JNK1 that has the following structure:

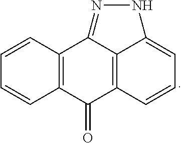 [SMILES: O=C1c2ccccc2-c2n[nH]c3cccc1c23]

By “U0126” is meant an inhibitor of mitogen activated ERK kinase (MEK) that has the following structure:

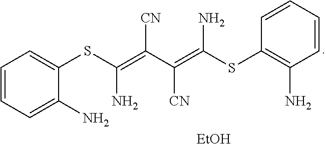 [SMILES: "EtOH" ; N#CC(=C(/N)Sc1ccccc1N)/C(C#N)=C(\N)Sc1ccccc1N]

By “Angiotensin II Receptor Type 2 (AT2)” is meant a protein that is encoded by the AGTR2 gene. AT2 is a G protein-coupled receptor that is activated by angiotensin II.

By “Fibrillin 1 gene” or “FBN1” is meant the gene located on the long arm of chromosome 15 at 15q21.1 (molecular location on chromosome 15: base pairs 48,700,502 to 48,937,984) that encodes the protein fibrillin-1. Fibrillin-1 is a component of the extracellular matrix. Marfan syndrome is caused by mutations in FBN1.

By “agent” is meant any small molecule chemical compound, antibody, nucleic acid molecule, or polypeptide, or fragments thereof.

By “ameliorate” is meant decrease, suppress, attenuate, diminish, arrest, or stabilize the development or progression of a disease.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.”

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of the analyte to be detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition or disorder that damages or interferes with the normal function of a cell, tissue, or organ. Examples of diseases include Marfan Syndrome.

By “effective amount” is meant the amount of a required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

The invention provides a number of targets that are useful for the development of highly specific drugs to treat or a disorder characterized by the methods delineated herein. In addition, the methods of the invention provide a facile means to identify therapies that are safe for use in subjects. In addition, the methods of the invention provide a route for analyzing virtually any number of compounds for effects on a disease described herein with high-volume throughput, high sensitivity, and low complexity.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “inhibitory nucleic acid” is meant a double-stranded RNA, siRNA, shRNA, or antisense RNA, or a portion thereof, or a mimetic thereof, that when administered to a mammalian cell results in a decrease (e.g., by 10%, 25%, 50%, 75%, or even 90-100%) in the expression of a target gene. Typically, a nucleic acid inhibitor comprises at least a portion of a target nucleic acid molecule, or an ortholog thereof, or comprises at least a portion of the complementary strand of a target nucleic acid molecule. For example, an inhibitory nucleic acid molecule comprises at least a portion of any or all of the nucleic acids delineated herein.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any protein or polynucleotide having an alteration in expression level or activity that is associated with a disease or disorder.

As used herein, “obtaining” as in “obtaining an agent” includes synthesizing, purchasing, or otherwise acquiring the agent.

“Primer set” means a set of oligonucleotides that may be used, for example, for PCR. A primer set would consist of at least 2, 4, 6, 8, 10, 12, 14, 16, 18, 20, 30, 40, 50, 60, 80, 100, 200, 250, 300, 400, 500, 600, or more primers.

By “reduces” is meant a negative alteration of at least 10%, 25%, 50%, 75%, or 100%.

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween. By “siRNA” is meant a double stranded RNA. Optimally, an siRNA is 18, 19, 20, 21, 22, 23 or 24 nucleotides in length and has a 2 base overhang at its 3′ end. These dsRNAs can be introduced to an individual cell or to a whole animal; for example, they may be introduced systemically via the bloodstream. Such siRNAs are used to downregulate mRNA levels or promoter activity.

By “specifically binds” is meant a compound or antibody that recognizes and binds a polypeptide of the invention, but which does not substantially recognize and bind other molecules in a sample, for example, a biological sample, which naturally includes a polypeptide of the invention.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507). For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 .mu.g/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant a mammal, including, but not limited to, a human or non-human mammal, such as a bovine, equine, canine, ovine, or feline.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### DESCRIPTION OF THE DRAWINGS

FIGS. 1A-1D are diagrams and graphs demonstrating the role of AngII in the aorta. FIG. 1A shows that AngII acts on the AT1 receptor causing increased cellular proliferation, fibrosis, and matrix metalloproteinase-2 and/or -9 (MMP2/9) activity while decreasing apoptosis. Conversely, the AT2 receptor is thought to decrease proliferation, fibrosis, and MMP activity, while increasing apoptosis. ACEi's block the conversion of AngI to AngII, limiting the signaling through both AT1 and AT2 receptors, whereas ARBs selectively block AT1. FIG. 1B shows the average absolute aortic root diameter (+/−2 SEM) measured serially by echocardiogram over the first year of life. Note that AT2KO:Fbn1^{C1039G/+} mice have a significantly larger aortic root diameter than Fbn1^{C1039G/+} mice at each time point. FIG. 1C is a Kaplan-Meier survival curve demonstrating an increased rate of death in AT2KO:Fbn1^{C1039G/+} mice as compared with Fbn1^{C1039G/+} mice. FIG. 1D is a graph showing ascending aortic growth from 2 to 12 months of age. Note the increased rate of ascending aortic growth in AT2KO:Fbn1^{C1039G/+} mice. Final absolute ascending aortic diameter: WT (1.41+/−0.07 mm), AT2KO (1.40+/−0.07 mm), Fbn1^{C1039G/+} (1.42+/−0.20 mm), AT2KO:Fbn1^{C1039G/+} (1.72+/−0.42 mm). In FIGS. 1B to 1D WT (n=5), AT2KO (n=10), Fbn1^{C1039G/+} (n=17), AT2KO:Fbn1^{C1039G/+} (n=19), *P<0.05; †P<0.001; ††P<0.0001; NS, not significant.

FIGS. 2A-2C show the therapeutic effects in the aorta AT2KO of losartan and enalapril. FIG. 2A are a panel of photomicrographs showing Verhoeff-Van Gieson (VVG) stain reveals diffuse fragmentation of elastic fibers and thickening of the media in Fbn1^{C1039G/+} mice; these finding are exaggerated in AT2KOFbn1^{C1039G/+} mice. WT (n=5), AT2KO (n=4), Fbn1^{C1039G/+} (n=7), and AT2KO:Fbn1^{C1039G/+} (n=7) mice. FIG. 2B is a graph showing average aortic root growth (+/−2 SEM) over 7 months of treatment in placebo- (n=13) or losartan- (n=7) treated WT mice and placebo- (n=17), losartan- (n=5), or enalapril- (n=15) treated Fbn1^{C1039G/+} mice, as measured by echocardiography. Note the regression in aortic size observed in losartan-treated Fbn1^{C1039G/+} mice and the marginal (P=0.05) decrease in growth in the enalapril-treated cohort. Final absolute aortic root diameter: WT (1.74+/−0.10 mm), losartan-treated WT (1.77+/−0.15 mm), Fbn1^{C1039G/+} (2.19+/−0.19 mm), losartan-treated Fbn1^{C1039G/+} (1.96+/−0.09 mm), and enalapril-treated Fbn1^{C1039G/+} (2.18+/−0.18). FIG. 2C is a graph showing average aortic root growth (+/−2 SEM) over 7 months of treatment in WT (n=8), placebo- (n=22), and losartan- (n=6) treated Fbn1^{C1039G/+} mice and placebo- (n=19), and losartan- (n=6) treated AT2KO:Fbn1^{C1039G/+} mice. Note the diminished effectiveness of losartan treatment in AT2KO:Fbn1^{C1039G/+} mice, as compared with losartan treatment in Fbn1^{C1039G/+} mice. Final absolute aortic root diameter: WT (1.77+/−0.10 mm), Fbn1^{C1039G/+} (2.13+/−0.16 mm), AT2KO:Fbn1^{C1039G/+} (2.34+/−0.13 mm), losartan-treated Fbn1^{C1039G/+} (1.96+/−0.09 mm), and losartan-treated AT2KO:Fbn1^{C1039G/+} (2.06+/−0.07 mm). In FIGS. 1B to 1D WT (n=5), AT2KO (n=10), Fbn1^{C1039G/+} (n=17), AT2KO:Fbn1^{C1039G/+} (n=19), *P<0.05; **P<0.01 †P<0.001; ††P<0.0001; NS, not significant.

FIG. 3 is a graph showing the measurement of average thickness (+/−2 SEM) of the proximal ascending aortic media from four representative sections of each mouse in WT (n=5), AT2KO (n=4), Fbn1^{C1039G/+} (n=7), and AT2KO:Fbn1^{C1039G/+} (n=7) mice. Note that AT2KO:Fbn1^{C1039G/+} mice show increased thickness, when compared to Fbn1^{C1039G/+} mice. *P<0.05; †P<0.001; NS, not significant.

FIG. 4 is graph showing quantification of elastic fiber content in WT (n=5), AT2KO (n=4), Fbn1^{C1039G/+} (n=7), and AT2KO:Fbn1^{C1039G/+} (n=7) mice reveals a reduction in Fbn1^{C1039G/+} mice, compared to WT mice, with a further decrease noted in AT2KO:Fbn1^{C1039G/+} mice. †P<0.001; NS, not significant.

FIG. 5 is graph showing average aortic wall architecture score (+/−2 SEM) of the proximal ascending aorta in WT (n=5), AT2KO (n=4), Fbn1^{C1039G/+} (n=7), and AT2KO:Fbn1^{C1039G/+} (n=7) mice. Note the greater aortic architecture score in Fbn1^{C1039G/+} mice compared to WT mice, and the exaggeration in AT2KO:Fbn1^{C1039G/+} mice. †P<0.001; NS, not significant.

FIG. 6 is a set of photomicrogaphs showing hematoxylin and eosin staining of the lung demonstrates diffuse distal airspace widening in Fbn1^{C1039G/+} mice compared to WT littermates; distal airspace caliber is further increased in AT2KO:Fbn1^{C1039G/+} mice. All images are shown at 10× magnification.

FIG. 7 is a graph showing average mean linear intercept (MLI; +/−2 SEM), a measure of distal airspace caliber, in WT (n=3), AT2KO (n=3), Fbn1^{C1039G/+} (n=4), and AT2KO:Fbn1^{C1039G/+} (n=5) mice. Note the significant increase in MLI in Fbn1^{C1039G/+} mice compared to WT littermates, and the further increase in AT2KO:Fbn1^{C1039G/+} animals. *P<0.05; NS, not significant.

FIG. 8 is a graph showing average systolic blood pressure (+/−2 SEM) in placebo- (n=4), losartan- (n=6) and enalapril- (n=8) treated Fbn1^{C1039G/+} mice, and placebo- (n=4), losartan- (n=6) and enalapril- (n=8) treated AT2KO:Fbn1^{C1039G/+} mice. Note that losartan- and enalapril-treated Fbn1^{C1039G/+} mice showed an equal reduction in systolic blood pressure when compared to placebo-treated animals. There was no significant difference in blood pressure between placebo-treated Fbn1^{C1039G/+} and AT2KO:Fbn1^{C1039G/+} mice., while losartan lowered blood pressure equally in Fbn1^{C1039G/+} and AT2KO:Fbn1^{C1039G/+} animals. †P<0.001; ††P<0.0001; NS, not significant.

FIG. 9 is a graph showing average aortic wall architecture (+/−2 SEM) in WT (n=5), placebo- (n=17), losartan- (n=5) and enalapril- (n=15) treated Fbn1^{C1039G/+} mice. Note that enalapril treatment was no more effective than placebo in Fbn1^{C1039G/+} mice, but significant improvement was seen in losartan-treated animals. *P<0.05; NS, not significant.

FIGS. 10A-10D show the mechanism of protection by AT2 signalling. FIG. 10A is a Western blot analysis of ERK1/2 and Smad2 activation in the aortic root and proximal ascending aorta of four mice of each genotype. Note that Smad2 activation is increased equally in AT2KO: Fbn1^{C1039G/+} and Fbn1^{C1039G/+} mice, compared with WT littermates. ERK1/2 activation is significantly increased in Fbn1^{C1039G/+} mice when compared with WT littermates and is further increased in AT2KO:Fbn1^{C1039G/+} mice. FIG. 10B is western blot analysis of ERK1/2 and Smad2 activation in the aortic root and proximal ascending aortas of three each of WT and placebo-, losartan- or enalapril-treated Fbn1^{C1039G/+} mice. Note that Smad2 activation is decreased in both losartan- and enalapril-treated Fbn1^{C1039G/+} mice when compared with placebo-treated animals, with a more pronounced effect in enalapril-treated animals. In contrast, enalapril treatment failed to reduce ERK1/2 activation, whereas losartan reduced ERK1/2 activation to levels indistinguishable from WT littermates. FIG. 10C is western blot analysis of ERK1/2 activation in the aortic root and proximal ascending aorta of three WT, AT2KO, and placebo- or losartan-treated AT2KO:Fbn1^{C1039G/+} mice. Note that losartan loses its ability to decrease ERK1/2 activation in AT2KO: Fbn1^{C1039G/+} mice, demonstrating that the inhibition of ERK1/2 activation is mediated by the AT2 receptor. FIG. 10D shows a summary of the effects of AngII receptors on both canonical and noncanonical TGFβ signaling. AT1 receptor stimulation drives ERK1/2 activation, whereas AT2 receptor stimulation inhibits it. Losartan attenuates ERK1/2 activation by blocking the AT1 cascade while simultaneously shunting signaling through the AT2 receptor. *P<0.05; **P<0.01 †P<0.001; NS, not significant.

FIG. 11 is a western blot analysis of ERK1/2 activation in the ascending (n=4) or descending (n=3) aorta in WT, AT2KO, Fbn1^{C1039G/+}, and AT2KO:Fbn1^{C1039G/+} mice. Note that in the ascending aorta, ERK1/2 activation is increased in Fbn1^{C1039G/+} mice, and is further increased in AT2KO:Fbn1^{C1039G/+} mice. In contrast, in the descending aorta, there is no significant difference in ERK1/2 activation in any of the genotypes. **P<0.01 †P<0.001; NS, not significant.

FIG. 12 is a western blot analysis of pJNK1 and pp 38 in the proximal ascending aorta of four WT, AT2KO, Fbn1^{C1039G/+} and AT2KO:Fbn1^{C1039G/+} mice. Note that there is no significant difference in pJNK1 or pp 38 between any of the mice. NS not significant.

FIG. 13 is a western blot analysis of pJNK1 and pp 38 in the proximal ascending aorta of three placebo-treated WT, and three placebo-, losartan- and enalapril-treated Fbn1^{C1039G/+} mice. Note that there is no significant difference in JNK1 or p38 activation in Fbn1^{C1039G/+} mice compared to WT controls; losartan or enalapril do not affect p38 activation, but both cause a small reduction in JNK1 activation. *P<0.05; **P<0.01; NS, not significant.

FIG. 14 is a western blot analysis of ERK1/2, Smad2, pJNK1 and p38 activation in three losartan-treated Fbn1^{C1039G/+} mice and three losartan-treated AT2KO:Fbn1^{C1039G/+} mice. Note that losartan's ability to inhibit ERK1/2 activation is lost in the absence of the AT2 receptor, while there is no significant change in Smad2, JNK1, or p38 activation. **P<0.01; NS, not significant.

FIG. 15 is a graph showing average aortic root growth (+/−2 SEM) over 7 months of treatment in WT (n=15), placebo- (n=17) and spironolactone-treated (n=5) Fbn1^{C1039G/+} mice, as measured by echocardiography. Note that there is no significant difference between placebo and spironolactone-treated Fbn1^{C1039G/+} animals. Final absolute aortic root diameter: WT 1.77+/−0.09, placebo-2.14+/−0.16, spironolactone-treated 2.19+/−0.19 Fbn1^{C1039G/+} mice. *P<0.05; **P<0.01; NS, not significant.

FIGS. 16A-16C are Western blots and corresponding graphs showing canonical and noncanonical TGFβ signaling in the proximal ascending aorta. FIG. 16A shows Western blot analysis of 4 WT and Fbn1^{C1039G/+} mice. Note that only pSmad2, ERK1/2, and pMEK1 signaling are increased in Fbn1^{C1039G/+} mice. The graphs show normalization to β-actin, but the same outcomes were observed with normalization to the respective total proteins. FIG. 16B shows Western blot analysis of three each of WT and of Fbn1^{C1039G/+} mice treated with placebo, TGF βNAb (Nab) or losartan (Los). FIG. 16C shows aortic root growth in placebo-treated WT (n=5), placebo-treated Fbn1^{C1039G/+} (n=6), RDEA119-treated WT (n=3), and RDEA119-treated Fbn1^{C1039G/+} (n=7) mice. Note that RDEA119 therapy selectively reduced growth in Fbn1^{C1039G/+} mice. Final absolute aortic root diameter (mm): WT (1.62+/−0.08), placebo-treated Fbn1^{C1039G/+} (2.15+/−0.17), RDEA119-treated WT (1.64+/−0.09), and RDEA119-treated Fbn1^{C1039G/+} (1.94+/−0.07). FIG. 16D shows Western blot analysis of three placebo- and three RDEA119-treated Fbn1^{C1039G/+} mice, showing a selective reduction in pERK1/2 signaling in RDEA119-treated mice. Plac, placebo. Values are means+/−2 SEM. *P<0.05; **P<0.01; t P<0.001; NS, not significant.

FIG. 17 is a Western blot analysis of the proximal ascending aorta of WT and Fbn1^{C1039G/+} mice. Note that there is no difference in either pERK1/2 or ROCK1 when normalized to β-actin. Values are the Mean+/−2 SEM. NS non-significant.

FIG. 18 is a graph showing aortic root growth over 4 months, measured by echocardiography, in placebo-treated WT (n=10) and Fbn1^{C1039G/+} (n=8) mice, and fasudil-treated WT (n=5) and Fbn1^{C1039G/+} (n=6) mice. Note the lack of rescue of aortic root growth in fasudil-treated Fbn1^{C1039G/+} mice. Absolute final aortic root diameter: placebo-treated WT (1.71+/−0.06), placebo-treated Fbn1^{C1039G/+} (2.19+/−0.18), fasudil-treated WT (1.73+/−0.05), fasudil-treated Fbn1^{C1039G/+} (2.40+/−0.16). Values are Mean+/−2 SEM. *P<0.05; †P<0.001; NS, not significant.

FIG. 19 is a graph showing aortic root growth over 2 months, measured by echocardiography, in placebo-treated WT (n=6) and Fbn1^{C1039G/+} (n=5) mice, and TGFβNAb-treated Fbn1^{C1039G/+} (n=4) mice. Note the full rescue of aortic root growth in TGFβNAb-treated Fbn1^{C1039G/+} mice. Final absolute aortic root diameter: placebo-treated WT (1.66+/−0.06), placebo-treated Fbn1^{C1039G/+} (2.19+/−0.18), TGFβNAb-treated Fbn1^{C1039G/+} (1.96+/−0.6). Values are Mean+/−2 SEM. **P<0.01; NS, not significant.

FIGS. 20A-20C show the effect of Smad4 haploinsufficiency (S4^{+/-}) on aortic phenotype. FIG. 20A is a survival curve of WT (n=112), S4^{+/-} (n=56), Fbn1^{C1039G/+} (n=107), and S4^{+/-}:Fbn1^{C1039G/+} (n=85) mice. Note the high rate of premature death due to aortic dissection in S4^{+/-}:Fbn1^{C1039G/+} mice. FIG. 20B shows aortic root and ascending aortic diameter, measured by echocardiography, at three months of age in WT (n=9), S4^{+/-} (n=11), Fbn1^{C1039G/+} (n=24), and S4^{+/-}:Fbn1^{C1039G/+} (n=26) mice. Although Fbn1^{C1039G/+} mice showed a selective increase in aortic root diameter compared with WT littermates, S4^{+/-}:Fbn1^{C1039G/+} mice demonstrated an increase in both aortic root and ascending aortic diameter, compared with all other genotypes. FIG. 20C is a panel of photomicrographs of VVG staining of representative sections of the proximal ascending aorta. Compared with WT littermates, Fbn1^{C1039G/+} mice demonstrated medial thickening and elastic fiber fragmentation, both of which are exacerbated in S4^{+/-}:Fbn1^{C1039G/+} mice. Values are means+/−2 SEM. †P<0.001; ††P<0.0001; NS, not significant.

FIG. 21 is a graph showing aortic architecture score in WT (n=12), S4^{+/-} (n=10), :Fbn1^{C1039G/+} (n=8), 16 and S4^{+/-}:Fbn1^{C1039G/+} (n=10) mice. While Fbn1^{C1039G/+} mice are worse that WT mice, there is an exacerbation in S4^{+/-}:Fbn1^{C1039G/+} animals. Values are Mean+/−2 SEM. *P<0.05; ††P<0.0001; NS, not significant.

FIG. 22 shows the effect of Smad4 haploinsufficiency (S4^{+/-}) on aortic signaling. Western blot analysis of the proximal ascending aorta in three mice each: WT, S4^{+/-}, Fbn1^{C1039G/+}, and S4^{+/-}:Fbn1^{C1039G/+}. Note the unique activation of JNK1 in S4^{+/-}:Fbn1^{C1039G/+} mice compared with all other genotypes. Values are Means+/−2 SEM. *P<0.05; **P<0.01; NS, not significant.

FIGS. 23A & 23B show the effect of JNK antagonism in the presence of SP600125. FIG. 23A shows aortic root and ascending aortic growth, as measured by echocardiography, in WT mice (n=6) and Fbn1^{C1039G/+} placebo- (n=5) or SP600125-treated (n=5) mice, as well as placebo- (n=8) or SP̂00125-treated (n=11) S4^{+/-}:Fbn1^{C1039G/+} littermates. Note that JNK inhibition decreased aortic root growth in S4^{+/-}:Fbn1^{C1039G/+} and Fbn1^{C1039G/+} mice and reduced ascending aortic growth in S4^{+/-}:Fbn1^{C1039G/+} mice. Final absolute aortic root and ascending aortic diameter (mm): WT (1.66+/−0.06; 1.33+/−0.06), placebo- (2.31+/−0.02; 1.43+/−0.10) or SP600125-treated (1.97+/−0.16; 1.38+/−0.06) Fbn1^{C1039G/+} mice, placebo- (2.33+/−0.38; 1.85+/−0.37) or SP̂00125-treated (2.09+/−0.16; 1.47+/−0.14) S4^{+/-}:Fbn1^{C1039G/+} mice. FIG. 23B is a graph of the survival curve for S4^{+/-}:Fbn1^{C1039G/+} mice treated with either placebo (n=8) or SP600125 (n=11), showing prevention of premature death in SP600125-treated animals. JNKi, JNK inhibitor SP600125; Plac, placebo. Values are the Mean+/−2 SEM. *P<0.05; **P<0.01 †P<0.001; ††P<0.0001; NS, not significant.

FIG. 24 shows Western blot analysis of the proximal ascending aorta of WT and Fbn1^{C1039G/+} mice after two weeks of therapy with SP600125 or placebo. While there is significant reduction in JNK1 activation in SP600125-treated animals to levels below baseline, there is no change in ERK1/2 activation. All values normalized to GAPDH. Values are Mean+/−2 SEM. **P<0.01 †P<0.001; NS, not significant.

FIG. 25 is a graph of the weight of placebo-treated WT (n=5) and Fbn1^{C1039G/+} (n=6) mice, and RDEA119-treated WT (n=3) and Fbn1^{C1039G/+} (n=7) mice, at the end of the two month trial. Note that RDEA119 treatment does not significantly affect somatic growth in either WT or Fbn1^{C1039G/+} mice. Values are Mean+/−2 SEM. NS, not significant.

FIG. 26 is a panel of photomicrographs of trichrome staining of representative proximal ascending aortic sections, showing increased collagen deposition in Fbn1^{C1039G/+} and S4⁺¹⁻:Fbn1^{C1039G/+} mice, compared to WT littermates. Note the collagen content is comparable in Fbn1^{C1039G/+} and S4^{+/-}:Fbn1^{C1039G/+} mice.

### DETAILED DESCRIPTION OF THE INVENTION

The instant invention is based on the discovery that loss of AT2 expression accelerates the aberrant growth and rupture of the aorta in a mouse model of Marfan syndrome (MFS). The selective AT1 receptor blocker (ARB) losartan abrogated aneurysm progression in the mice; full protection required intact AT2 signaling. The angiotensin-converting enzyme inhibitor (ACEi enalapril, which Limits signaling through both receptors, was less effective. Both drugs attenuated canonical transforming growth factor-β (TGFβ) signaling in the aorta, but losartan uniquely inhibited TGFβ-mediated activation of extracellular signal-regulated kinase (ERK), by allowing continued signaling through AT2. The invention highlights the protective nature of AT2 signaling and inform the choice of therapies in MFS and related disorders. Accordingly, the invention features agents that stimulate AT2 signaling, for example AT2 agonists.

Transforming growth factor-β (TGFβ) signaling drives aneurysm progression in multiple disorders, including Marfan syndrome (MFS), and therapies that inhibit this signaling cascade are in clinical trials. TGFβ can stimulate multiple intracellular signaling pathways, but it is unclear which of these pathways drives aortic disease and, when inhibited, which result in disease amelioration. The invention is based in part on the finding that extracellular signal-regulated kinase (ERK) 1 and 2 and Smad2 are activated in a mouse model of MFS, and both are inhibited by therapies directed against TGFβ. Whereas selective inhibition of ERK1/2 activation ameliorated aortic growth, Smad4 deficiency exacerbated aortic disease and caused premature death in MF5 mice. Smad4-deficient MFS mice uniquely showed activation of Jun N-terminal kinase-1 (JNK1), and a JNK antagonist ameliorated aortic growth in MFS mice that lacked or retained full Smad4 expression. Thus, noncanonical (Smad-independent) TGFβ signaling is a prominent driver of aortic disease in MFS mice, and inhibition of the ERK1/2 or JNK1 pathways is a potential therapeutic strategy for the disease.

### Marfan Syndrome (MFS)

Marfan syndrome (MFS) is an autosomal dominant connective tissue disorder that includes a predisposition for aortic root aneurysm and aortic rupture. MFS is caused by a deficiency of the microfibrillar constituent protein fibrillin-1 that is imposed by heterozygous mutations in FBN1. In prior work, we demonstrated that transforming growth factor-β (TGFβ) signaling was elevated in affected tissues of mice heterozygous for a cysteine substitution in an epidermal growth factor-like domain of fibrillin-1 (Fbn1^{C1039G/+}), the most common class of mutation in people with MFS (J. P. Habashi et al., Science 312, 117 (2006); E. R. Neptune et al., Nat. Genet. 33, 407 (2003); C. M. Ng et al., J. Clin. Invest. 114, 1586 (2004); and R. D. Cohn et al., Nat. Med. 13, 204 (2007)). Many disease manifestations—including aortic aneurysm (J. P. Habashi et al., Science 312, 117 (2006)), developmental emphysema (E. R. Neptune et al., Nat. Genet. 33, 407 (2003)), myxomatous degeneration of the atrioventricular valves (C. M. Ng et al., J. Clin. Invest. 114, 1586 (2004)), and skeletal muscle myopathy (R. D. Cohn et al., Nat. Med. 13, 204 (2007))—are attenuated by systemic administration of a pan-specific poly-clonal TGFβ-neutralizing antibody (TGFβNAb) in fibrillin-1-deficient mice. Similar protection was achieved by treating Fbn1^{C1039G/+} mice with the angiotensin II (AngII) type 1 (AT1) receptor blocker (ARB) losartan (J. P. Habashi et al., Science 312, 117 (2006); and R. D. Cohn et al., Nat. Med. 13, 204 (2007)). ARBs can attenuate TGFβ signaling in some tissues by lowering the expression of TGFβ ligands, receptors, and activators (G. Wolf, F. N. Ziyadeh, R. A. Stahl, J. Mol. Med. 77, 556 (1999); N. Fukuda et al., Am. J. Hypertens. 13, 191 (2000); and T. Naito et al., Am. J. Physiol. Renol Physiol. 286, F278 (2004)). In this mouse model of MFS, losartan's protection correlated with decreased phosphorylation and nuclear translocation of Smad2 (pSmad2), a direct effector of canonical TGFβ signaling, and decreased expression of prototypical Smad-dependent TGFβ-responsive gene products, such as connective tissue growth factor and collagens.

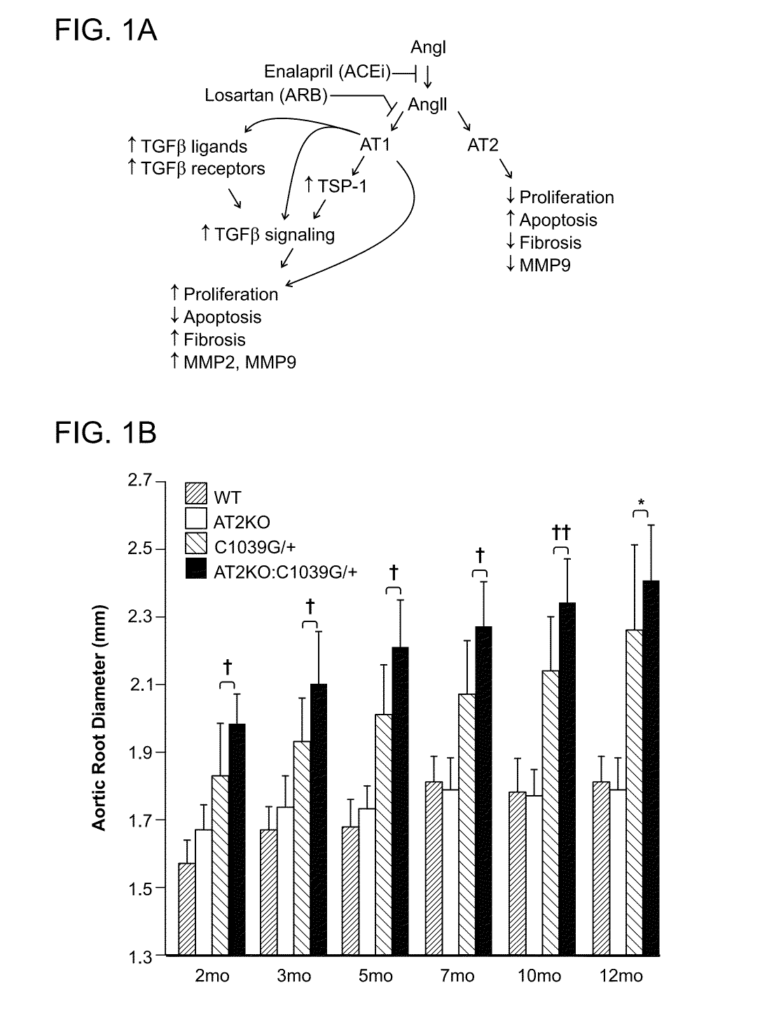

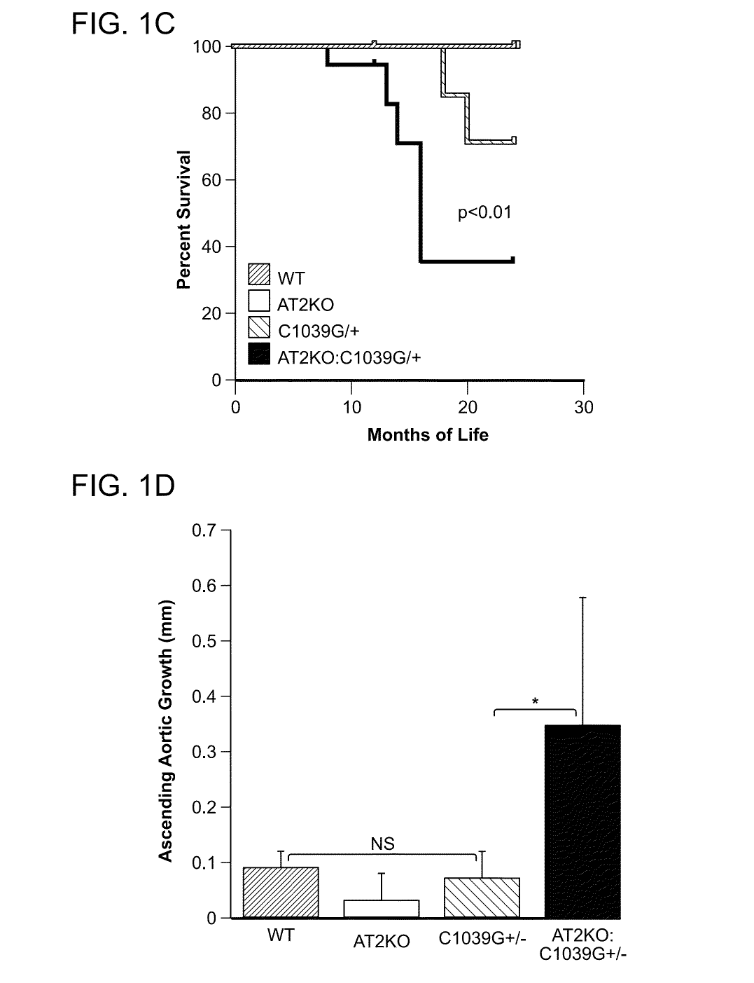

The contribution of AT2 to aortic aneurysm progression remains controversial. AT2 signaling can oppose AT1 mediated enhancement of TGFβ signaling in some cell types and tissues (FIG. 1A) (E. S. Jones, M. J. Black, R. E. Widdop, J. Mol. Cell. Cardial. 37, 1023 (2004); and J. Rodriguez-Vita et al., Circulation 111, 2509 (2005)). It can also induce vascular smooth muscle cell (VSMC) apoptosis, theoretically contributing to aortic wall damage. Apoptosis was observed in cultured cells derived from end-stage aneurysms in people with MFS(H. Nagashima et al., Circulation 104 (suppl. 1), 1282 (2001)), but has not been found in early- or intermediate-stage aortic wall lesions in MFS mice (J. P. Habashi et al., Science 312, 117 (2006)). Vascular expression of AT2 is largely limited to prenatal life, but it may continue to be relevant postnatally in the context of certain disease states, as evidenced by the acceleration of inflammatory aneurysms in AngII-infused mice treated with an AT2 antagonist (A. Daugherty, M. W. Manning, L. A. Cassis, Br. J. Pharmacol. 134, 865 (2001)). In contrast, β-aminopropionitrile monofumarate (BAPN) induced aortic aneurysm and dissection in rats, which was associated with increased expression of AT2 and VSMC apoptosis, was ameliorated by limiting AngII production with angiotensin-converting enzyme inhibitor (ACEi) but not by selective AT1 receptor blockade (H. Nagashima et al., J. Vasc. Surg. 36, 818 (2002)). AT2 signaling has the capacity to attenuate both canonical (Smad-dependent) and noncanonical (mitogenactivated protein kinase or MAPK) TGFβ signaling cascades, most notably the extracellular signal—regulated kinase (ERK), in some tissues (B. Ulmasov, Z. Xu. L. H. Tetri, T. Inagami, B. A. Neuschwander-Tetri, Am. J. Physiol. Gastrointest. Liver Physiol. 296, G284 (2009); and M. Akishita et al., J. Clin. Invest. 103, 63 (1999)). Thus, AT2 signaling can both augment and inhibit the pathogenesis of aneurysm in pre-clinical models, and the mechanistic explanation for the discordance is unclear. This has direct clinical relevance, as it leaves open to question the relative therapeutic merits of selective AT1 blockade with ARBs versus limiting signaling through both AT1 and AT2 with ACEi, despite small trials suggesting that either approach has potential in MFS (B. S. Brooke et al., N. Engl. J. Med. 358, 2787 (2008); A. T. Yetman, R. A. Bornemeier, B. W. McCrindle, Am. J. Cardiol. 95, 1125 (2005); and A. A. Ahimastos et al., JAMA 298, 1539 (2007)).

### Transforming Growth Factor β (TGFβ) Signaling

The transforming growth factor-β (TGFβ) ligands belong to a family of cytokines that regulates diverse cellular functions, including proliferation, differentiation, and synthetic repertoire. TGFβ is secreted from cells as part of a large latent complex that binds to extracellular matrix (ECM) proteins including fibrillin-1 (Z. Isogai et al., J. Biol, Chem. 278, 2750 (2003)), the deficient gene product in Marfan syndrome (MFS). Current models posit that ECM sequestration of TGFβ inhibits its activation, thereby limiting its ability to stimulate cell surface receptors, TβRI and TβRII (H. C. Dietz, J. Clin. Invest. 120, 403 (2010); and R. O. Hynes, Science 326, 1216 (2009)). In canonical signaling, the TβRI/II complex phosphorylates receptor-activated Smad2 and/or Smad3 (to pSmad2 and pSmad3, respectively), which leads to recruitment of Smad4, translocation to the nucleus, and the transcription of Smad-dependent genes (J. S. Kang, C. Liu, R. Derynck, Trends Cell Biol. 19, 385 (2009)). Recent work has shown that TGFβ also induces other (noncanonical) pathways, including the RhoA and mitogen-activated protein kinase (MAPK) cascades, the latter of which includes extracellular signal-regulated kinase (ERK), Jun N-terminal kinase (JNK), and p38 (R. Derynck, Y. E. Zhang, Nature 425, 577 (2003); M. K. Lee et al., EMBO J. 26, 3957 (2007); and M. Yamashita et al., Mol. Cell 31, 918 (2008)). TGFβ activates these by phosphorylation to pERK, pJNK, and pp38, respectively. In light of these findings, the exclusive focus on Smad signaling in TGFβ-related pathogenetic models needs to be reconsidered.

Increased Smad2/3 activation and increased expression of Smad-responsive genes (e.g., connective tissue growth factor and plasminogen-activator inhibitor-1 PAI-1) have been observed in the lung, skeletal muscle, mitral valve, and aortic wall in humans and a mouse model of MFS (E. R. Neptune et al., Nat. Genet. 33, 407 (2003); C. M. Ng et al., J. Clin. Invest. 114, 1586 (2004); J. P. Habashi et al., Science 312, 117 (2006); and R D. Cohn et al., Nat. Med. 13, 204 (2007)). Treatment of MFS mice with TGFβ-neutralizing antibody (TGFβNAb) ameliorates the phenotype in all of these tissues, in association with attenuated pSmad2/3 signaling (Id.). A similar rescue is achieved by using the angiotensin II type 1 receptor-blocker losartan (Id.), which is known to reduce the expression of TGFβ ligands, receptors, and activators (G. Wolf, F. N. Ziyadeh, R. A. Stahl, J. Mol. Med. 77, 556 (1999); N. Fukuda et al., Am. J. Hypertens. 13, 191 (2000); and T. Naito et al., Am. J. Physiol. Renal Physiol. 286, F278 (2004)). It has also been shown that mutations in WI or II, which lead to a paradoxical increase in pSmad2 signaling in the aortic wall, cause Loeys-Dietz syndrome, a condition that has considerable phenotypic overlap with MFS, including aortic aneurysm (B. L. Loeys et al., Nat. Genet. 37, 275 (2005); and B. L. Loeys et al., N. Engl. J. Med. 355, 788 (2006)). Together, these earlier observations suggested that canonical TGFβ signaling drives disease pathogenesis in MFS. We have now explored the relative contributions of canonical and noncanonical TGFβ signaling cascades in MFS mice, by either genetically or pharmacologically inhibiting each cascade and analyzing the resultant phenotypic consequences.

### Agents of the Invention

The invention provides agents to modulate the expression or activity of noncanonical TGFβ signaling pathways. In one embodiment, the agent is a TGFβ antagonist that selectively blocks TGFβ signaling pathways other than those mediated by Smad2/3. Agents that block upstream activators of ERK1/2 are examples of agents that block noncanonical TGFβ signaling. In a particular embodiment, the agent is an inhibitor of MEK, ERK1/2, or JNK1. Non-limiting illustrative examples include SP600125, RDEA119, and U0126.

As used herein, a “noncanonical TGFβ signaling inhibitor” is any molecule which is able to decrease the amount or activity of a noncanonical TGF-β signaling pathway, either within a cell or within a physiological system. Exemplary antagonists include compounds, molecules, or agents that inhibit a biological activity. Examples of antagonist molecules include, but are not limited to, peptides, small molecules, antibodies, antisense nucleic acids, siRNA nucleic acids, aptamers, and other binding agents. The ability to decrease the amount or activity of a noncanonical TGFβ signaling pathway is not limited by any mechanism. For example, a noncanonical TGFβ signaling inhibitor may be a molecule which inhibits expression of a component of the noncanonical TGFβ signaling pathway at the level of transcription, translation, processing, or transport. In preferred embodiments, noncanonical TGFβ signaling inhibitors are small molecules that inhibit a component member of the noncanonical TGFβ signaling pathway.

A variety of noncanonical TGFβ signaling inhibitors and methods for their production are well known in the art and many more are currently under development. The specific noncanonical TGFβ signaling inhibitor employed is not a limiting feature, as any effective noncanonical TGFβ signaling inhibitor may be useful in the methods of this invention.

Agents useful in the methods of the invention can be nucleic acid molecules, e.g., antisense, ribozyme, or RNA interference technology, e.g., siRNA molecules corresponding to a portion of the nucleotide sequence encoding a component member of the noncanonical TGFβ signaling pathway (e.g., a nucleic acid encoding ERK1/2).

Antisense polynucleotides may act by directly blocking translation by hybridizing to mRNA transcripts or degrading such transcripts of a gene. The antisense molecule may be recombinantly made using at least one functional portion of a gene in the antisense orientation as a region downstream of a promoter in an expression vector. Chemically modified bases or linkages may be used to stabilize the antisense polynucleotide by reducing degradation or increasing half-life in the body (e.g., methyl phosphonates, phosphorothioate, peptide nucleic acids). The sequence of the antisense molecule may be complementary to the translation initiation site (e.g., between −10 and +10 of the target's nucleotide sequence).

Ribozymes catalyze specific cleavage of an RNA transcript or genome. The mechanism of action involves sequence-specific hybridization to complementary cellular or viral RNA, followed by endonucleolytic cleavage. Inhibition may or may not be dependent on ribonuclease H activity. The ribozyme includes one or more sequences complementary to the target RNA as well as catalytic sequences responsible for RNA cleavage (e.g., hammerhead, hairpin, axehead motifs). For example, potential ribozyme cleavage sites within a subject RNA are initially identified by scanning the subject RNA for ribozyme cleavage sites which include the following trinucleotide sequences: GUA, GUU and GUC. Once identified, an oligonucleotide of between about 15 and about 20 ribonucleotides corresponding to the region of the subject RNA containing the cleavage site can be evaluated for predicted structural features, such as secondary structure, that can render candidate oligonucleotide sequences unsuitable. The suitability of candidate sequences can then be evaluated by their ability to hybridize and cleave target RNA. The ribozyme may be recombinantly produced or chemically synthesized.

siRNA refers to double-stranded RNA of at least 20-25 basepairs which mediates RNA interference (RNAi). Duplex siRNA corresponding to a target RNA may be formed by separate transcription of the strands, coupled transcription from a pair of promoters with opposing polarities, or annealing of a single RNA strand having an at least partially self-complementary sequence. Alternatively, duplexed oligoribonucleotides of at least about 21 to about 23 basepairs may be chemically synthesized (e.g., a duplex of 21 ribonucleotides with 3′ overhangs of two ribonucleotides) with some substitutions by modified bases being tolerated. Mismatches in the center of the siRNA sequence, however, abolishes interference. The region targeted by RNA interference should be transcribed, preferably as a coding region of the gene. Interference appears to be dependent on cellular factors (e.g., ribonuclease III) that cleave target RNA at sites 21 to 23 bases apart; the position of the cleavage site appears to be defined by the 5′ end of the guide siRNA rather than its 3′ end. Priming by a small amount of siRNA may trigger interference after amplification by an RNA-dependent RNA polymerase.

Pharmaceutical Compositions of the Invention

The agents described herein can be formulated into pharmaceutical compositions for the treatment of the diseases, disorders and conditions disclosed herein. The language “pharmaceutical composition” includes preparations suitable for administration to mammals, e.g., humans. When the compounds used in the methods of the present invention are administered as pharmaceuticals to mammals, e.g., humans, they can be given per se or as a pharmaceutical composition containing, for example, 0.1 to 99.5% (more preferably, 0.5 to 90%) of active ingredient in combination with a pharmaceutically acceptable carrier.

The phrase “pharmaceutically acceptable carrier” is art recognized and includes a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (13HT), lecithin, propyl gallate, .alpha.-tocopherol, and the like; and metal chelating agents, such as citric acid, ethylenediamine tetraacetic acid (EDTA), sorbitol, tartaric acid, phosphoric acid, and the like.

Formulations of the present invention include those suitable for oral, nasal, topical, transdermal, buccal, sublingual, rectal, vaginal and/or parenteral administration. The formulations may conveniently be presented in unit dosage form and may be prepared by any methods well known in the art of pharmacy. The amount of active ingredient that can be combined with a carrier material to produce a single dosage form will generally be that amount of the compound that produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol and glycerol monostearate; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluent commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert dilutents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the active compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue,

The preparations of the present invention may be given orally, parenterally, topically, or rectally. They are of course given by forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administration is preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracistemally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound that is the lowest dose effective to produce a therapeutic effect. Such an effective dose will generally depend upon the factors described above. Generally, intravenous and subcutaneous doses of the compounds of this invention for a patient, when used for the indicated analgesic effects, will range from about 0.0001 to about 100 mg per kilogram of body weight per day, more preferably from about 0.01 to about 50 mg per kg per day, and still more preferably from about 1.0 to about 100 mg per kg per day. An effective amount is that amount treats a disease, disorder or condition set forth herein.

If desired, the effective daily dose of the active compound may be administered as two, three, four, five, six or more sub-doses administered separately at appropriate intervals throughout the day, optionally, in unit dosage forms.

While it is possible for a compound of the present invention to be administered alone, it is preferable to administer the compound as a pharmaceutical composition.

Methods of Treatment

As used herein, the term “Marfan syndrome or associated diseases, disorders and conditions” is intended to mean Marfan syndrome or any one of the multitude of diseases disorders or conditions that is caused or associated with the biochemical events that cause Marfan syndrome, e.g., the aberrant expression or activity or TGFβ. Exemplary conditions include aneurysm, an aortic aneurysm, valve disease, emphysema, myopathy, scoliosis, or eye disease. Exemplary eye diseases include cataracts, myopia, glaucoma, and retinal detachment. Moreover, Marfan syndrome or associated diseases, disorders and conditions include diseases and disorders that related to muscle growth, maintenance, or regeneration, e.g., muscular dystrophies such as Duchenne muscular dystrophy. Further, the disease or disorder can be a lung disease or disorder, e.g., emphysema, pneumothorax, and COPD.

The term “treated,” “treating” or “treatment” includes the diminishment or alleviation of at least one symptom associated or caused by Marfan syndrome, or an associated disease, disorder or condition. For example, treatment can be diminishment of one or several symptoms of a disease or disorder or complete eradication of the disease or disorder, e.g., Marfan syndrome.

The term “subject” is intended to include organisms, e.g., prokaryotes and eukaryotes, which are capable of suffering from or afflicted with Marfan syndrome, or a disease, disorder or condition related thereto. Examples of subjects include mammals, e.g., humans, dogs, cows, horses, pigs, sheep, goats, cats, mice, rabbits, rats, and transgenic non-human animals. In certain embodiments, the subject is a human, e.g., a human suffering from, at risk of suffering from, or potentially capable of suffering from a Marfan syndrome, or a disease, disorder or condition related thereto.

The agents and pharmaceutical compositions of the invention can be administered to a subject to treat or prevent diseases, disorders and conditions associated with aberrant noncanonical TGFβ signaling. In one embodiment the agents and pharmaceutical compositions are used to treat or prevent Marfan syndrome or diseases or disorders associated with Marfan syndrome.

In one embodiment, the agents or pharmaceutical compositions are administered in an effective amount using a dosing schedule determined by a medical provider to treat or prevent a disease or disorder set forth herein. The agents or pharmaceutical compositions can be administered in a variety or methods described herein and known to one of skill in the art.

In one aspect, the invention provides a method for preventing in a subject, a disease or condition associated with an aberrant or unwanted noncanonical TGFβ signaling, by administering to the subject an agent which modulates noncanonical TGFβ signaling. Subjects at risk for a disease which is caused or contributed to by aberrant expression or activity of noncanonical TGFβ signaling can be identified by, for example, any or a combination of diagnostic or prognostic assays as described herein. Administration of a prophylactic agent can occur prior to the manifestation of symptoms characteristic of the noncanonical TGFβ signaling aberrancy, such that a disease or disorder is prevented or, alternatively, delayed in its progression.

Another aspect of the invention pertains to methods of modulating noncanonical TGFβ signaling for therapeutic purposes. Accordingly, in an exemplary embodiment, the modulatory method of the invention involves contacting a cell with an agent that modulates one or more of the activities of a component of a noncanonical TGFβ signaling pathway. An agent that modulates noncanonical TGFβ signaling activity can be an agent as described herein, such as a nucleic acid, a polypeptide, or a small molecule. In one embodiment, the agent inhibits one or more TGF-β activities. Examples of such inhibitory agents include antisense ERK1/2 nucleic acid molecules, anti-ERK1/2 antibodies, and ERK1/2 inhibitors. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant or unwanted noncanonical TGFβ signaling, e.g., Marfan syndrome or an associated disease or disorder. In one embodiment, the method involves administering an agent, or combination of agents that modulates noncanonical TGFβ signaling.

The invention further provides kits comprising agents or pharmaceutical compositions of the invention and instructions for use. In one embodiment, the kits of the invention are for the treatment of diseases and disorders characterized by aberrant noncanonical TGFβ signaling. In a related embodiment, the noncanonical TGFβ signaling associated disease or disorder is Marfan syndrome or a disease or disorder related to Marfan syndrome.

### EXAMPLES

It should be appreciated that the invention should not be construed to be limited to the examples that are now described; rather, the invention should be construed to include any and all applications provided herein and all equivalent variations within the skill of the ordinary artisan.

### Example 1

### AT2 Receptor Elimination Exacerbates Aortic Disease in MFS Mice

To assess the role of the AT2 receptor in MFS, mice with a disrupted Agtr2 allele (encoding AT2; AT2KO) (T. Ichiki et al., Nature 377, 748 (1995)) were bred with Fbn1^{C1039G/+} mice, an established model of MFS (D. P. Judge et al., J. Clin. Invest. 114, 172 (2004)). Agtr2 is encoded on the X chromosome in humans and mice, and the AT2KO allele associates with loss of mRNA and protein expression, as assessed by radioligand binding, in either homozygous females or hemizygous males. The AT2KO mice develop normally, with no evidence of cardiovascular pathology or early mortality (H. M. Siragy, T. Inagami, T. Ichiki, R. M. Carey, Proc. Natl. Acad. Sci. U.S.A. 96, 6506 (1999)).

The progression of aortic root aneurysm was followed by echocardiogram until the mice were killed at 12 months (FIG. 1B). There was a small difference in aortic root size between wild-type (WT) and AT2KO mice (P<0.05) at 2 months, but this difference was absent at all future time points (P=0.70). The aortic root diameter of AT2KO:Fbn1^{C1039G/+} mice was significantly larger than that seen in Fbn1^{C1039G/+} mice at 2 months of age (P<0.001), and this difference was maintained through to 12 months of life (P<0.05). The postnatal aortic root growth over 10 months was not different between Fbn1^{C1039G/+} mice with or without AT2 expression (P=0.80). This could reflect postnatal waning of AT2 receptor expression, attainment of an absolute threshold of aortic root growth rate in AT2KO:Fbn1^{C1039G/+} mice, and/or the accelerated death observed in AT2KO:Fbn1^{C1039G/+} mice that effectively removed the most severely affected animals from later analyses. 32% of AT2KO:Fbn1^{C1039G/+} mice died before the scheduled killing, compared with 12% of Fbn1^{C1039G/+} mice (P<0.01) and 0% of AT2KO or WT mice (FIG. 1C). Growth of the more distal ascending aorta over 10 months was significantly greater in AT2KO:Fbn1^{C1039G/+} mice compared with Fbn1^{C1039G/+} littermates (P<0.05), whereas there was no significant difference between WT, AT2KO, and Fbn1^{C1039G/+} mice (FIG. 1D).

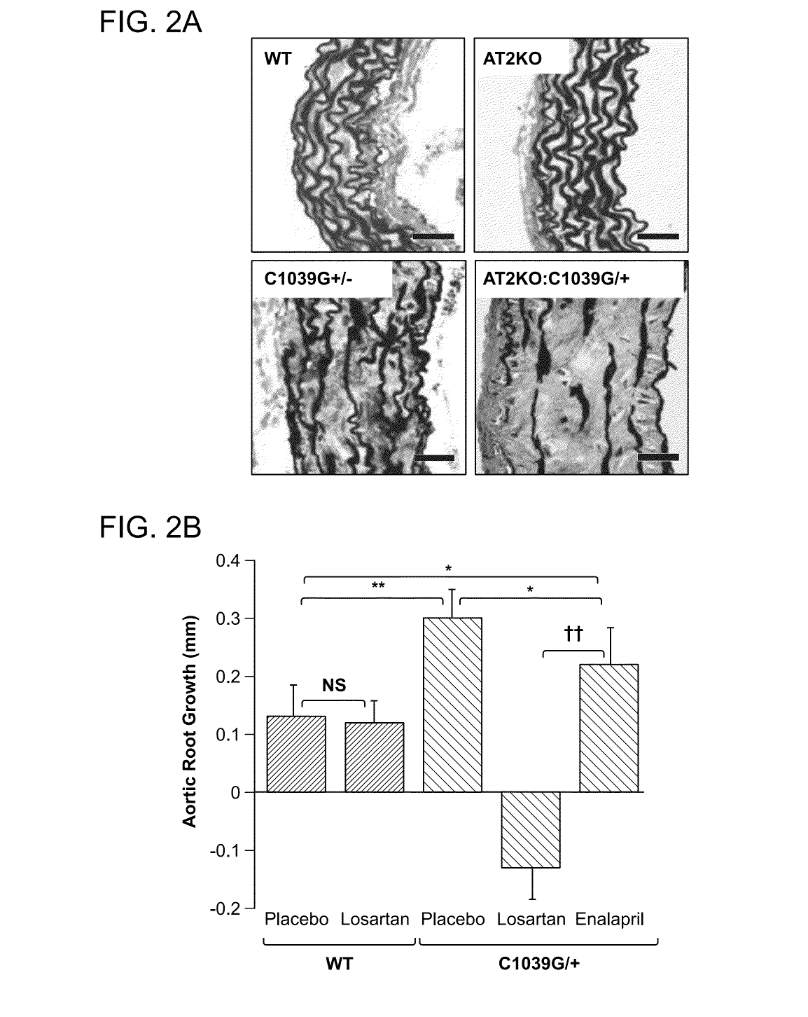

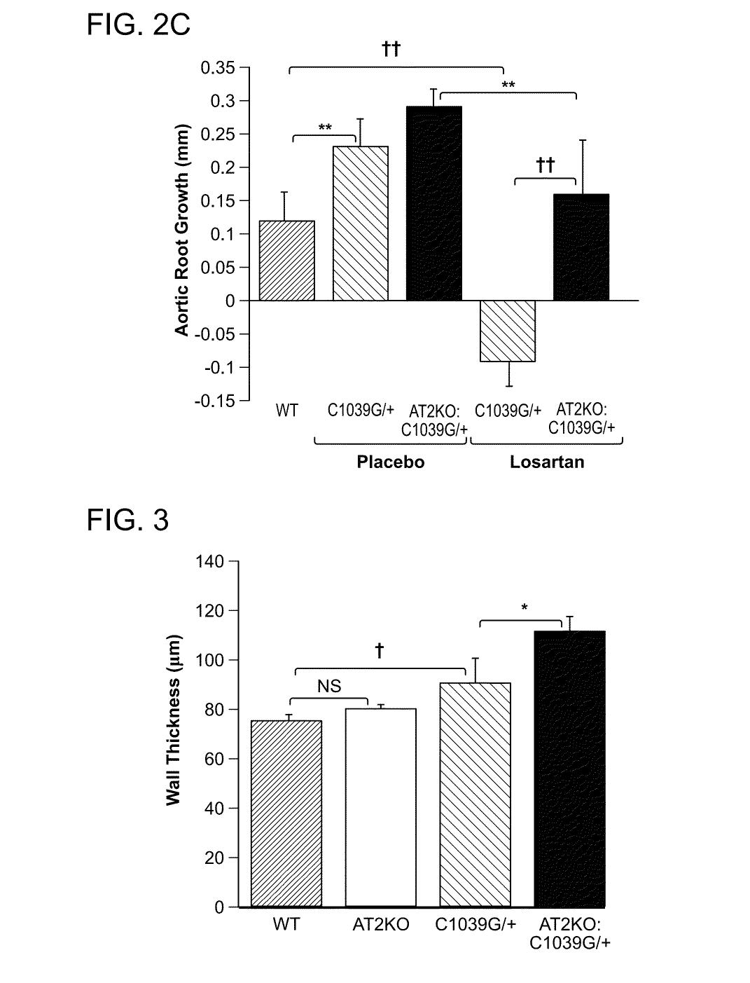

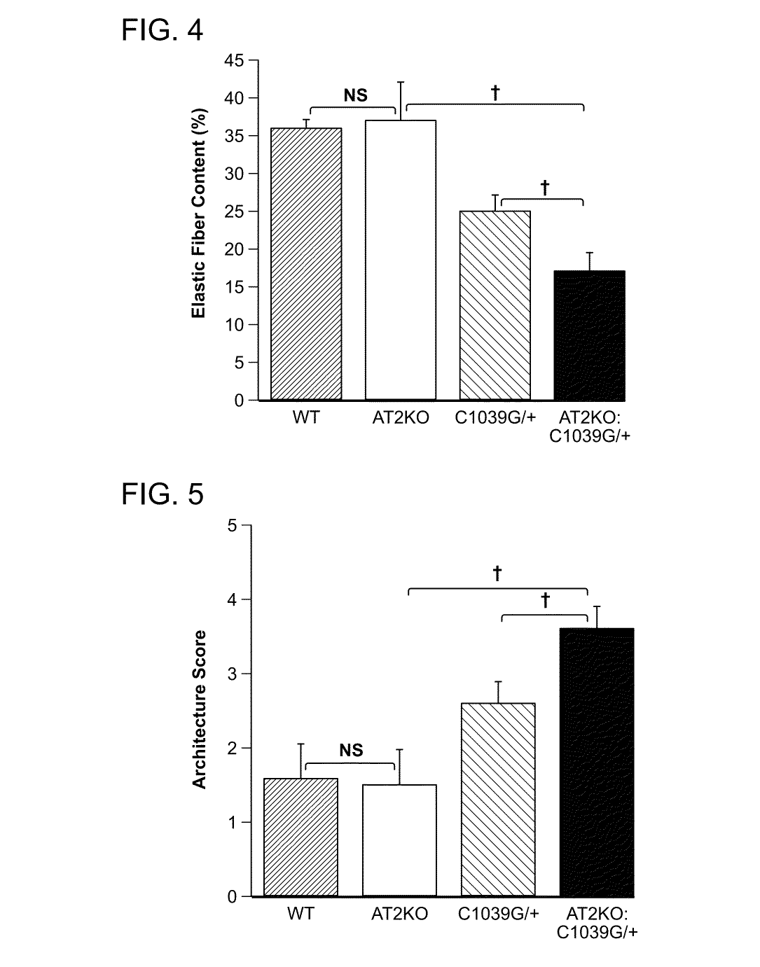

Histological and morphometric analyses of the aortic media were performed at 12 months. AT2KO:Fbn1^{C1039G/+} mice showed medial thickening, reduced elastin content, and increased elastic fiber fragmentation (FIGS. 2A, 3, 4, and 5) compared with Fbn1^{C1039G/+} or AT2KO mice (P<0.01 for all comparisons). These parameters were not significantly different in AT2KO and WT mice (P=0.07, P=0.68, and P=1.0, respectively). Therefore, the histological changes in the aorta paralleled the echocardiography findings, which supported the conclusion that AT2 receptor elimination exacerbates aortic disease in MFS mice.

### Example 2

### AT2 Receptor Elimination Exacerbates the MFS Phenotype Outside the Cardiovascular System

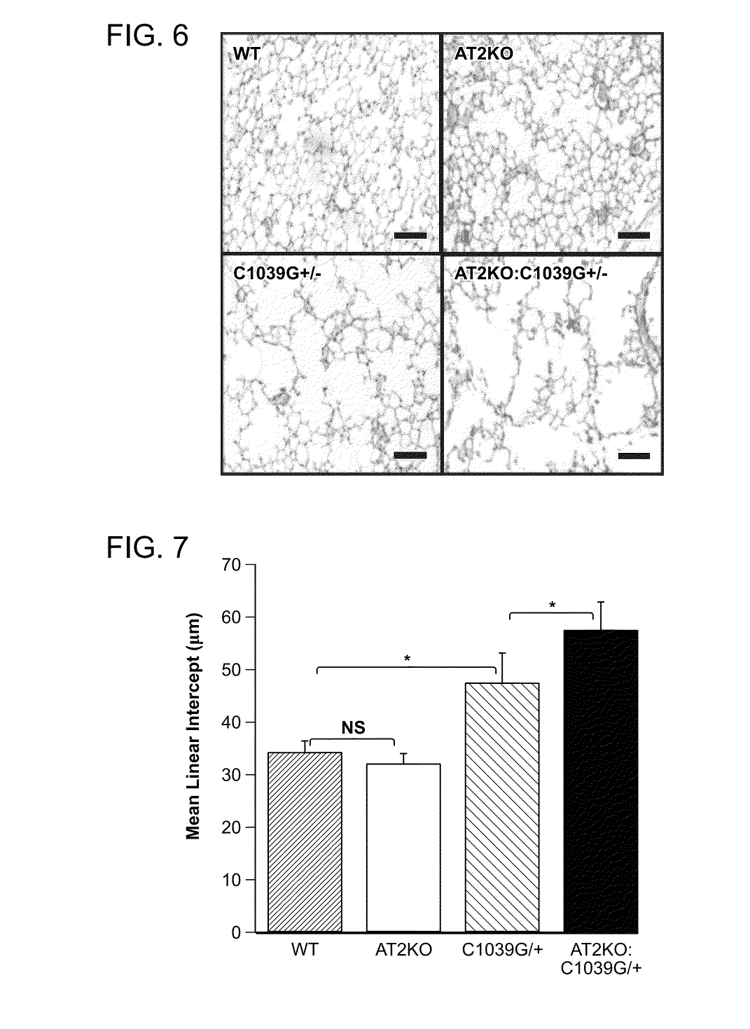

The potential for exacerbation of the MFS phenotype outside of the cardiovascular system was also assessed. At 12 months, excised lungs were inflated with agar, sectioned, and stained for histological and morphometric analyses (FIGS. 6 and 7). Increased distal airspace caliber, a marker of impaired distal alveolar septation and emphasematous lung disease, can be quantified by calculating a mean linear intercept (MLI). There was no significant difference in MLI between WT and AT2KO mice (P=1.0). Compared with WT and AT2KO littermates, Fbn1^{C1039G/+} mice had a significant increase in MLI (P<0.05), whereas Fbn1^{C1039G/+} mice had a yet further increase in MLI (P<0.05). This confirms that AT2 receptor elimination exacerbates the MFS phenotype outside of the cardiovascular system.

### Example 3

### AT2 Receptor Signaling Protectively Modifies MFS and is Needed to Achieve the Full Therapeutic Potential of ARBs

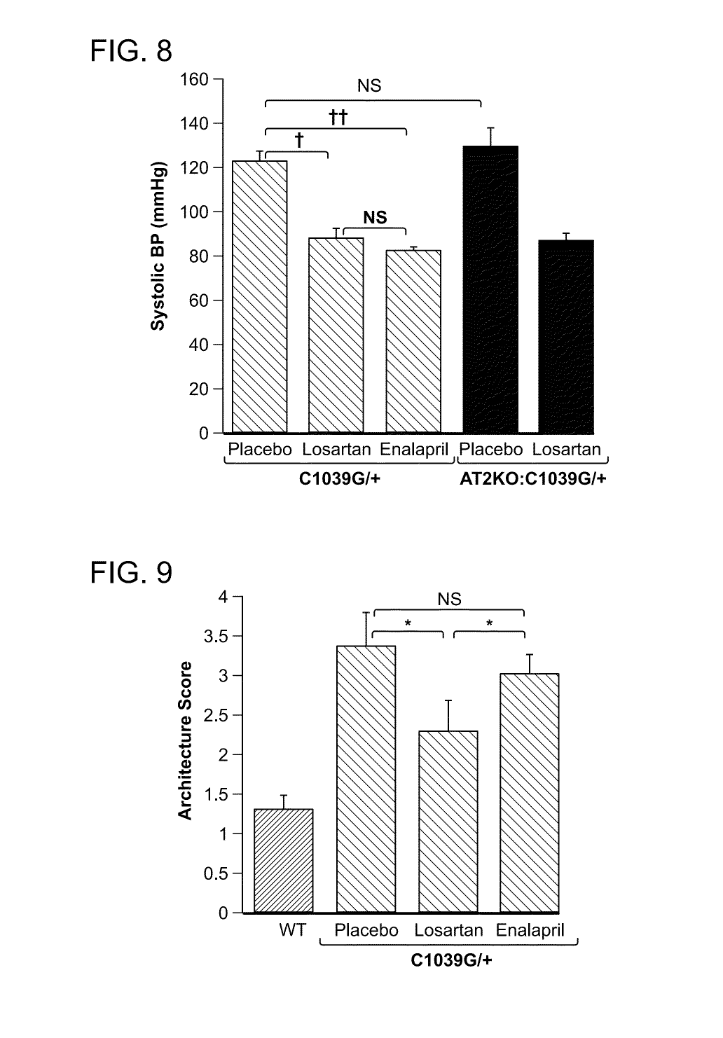

A head-to-head comparison of ACEi versus ARBs was performed. Fbn1^{C1039G/+} mice and WT littermates were treated with hemodynamically equivalent doses (FIG. 8) of either the ACEi enalapril (10 to 15 mg/kg of body weight per day) or the ARB losartan (40 to 60 mg/kg per day) (R. D. Patten et al., Clin. Sci 104, 109 (2003)), beginning at 8 weeks of age, and were assessed with serial echocardiograms. Aortic root growth over the 7 months of treatment was significantly greater in placebo-treated Fbn1^{C1039G/+} mice compared with WT littermates (P<0.01), whereas losartan led to a significant regression in growth in Fbn1^{C1039G/+} mice (P<0.0001), to rates that were significantly less than that seen in WT littermates (P<0.0001) (J. P. Habashi et al., Science 312, 117 (2006)). It is noteworthy that losartan reduced aortic root growth in Fbn1^{C1039G/+} mice, but had no effect in WT littermates (P=0.27). Enalapril treatment had significantly less effect than losartan in Fbn1^{C1039G/+} mice (P<0.0001); in fact, it was only marginally better than placebo treatment (P=0.05) (FIG. 2B). Enalapril was also no more beneficial than placebo in improving aortic architecture score in Fbn1^{C1039G/+} mice (P=0.19), whereas losartan was significantly more beneficial than both placebo and enalapril treatment (P<0.05 for both) (FIG. 9). Whether AT2 signaling is needed to achieve losartan's full therapeutic benefit was assessed. AT2KO:Fbn1^{C1039G/+} mice were treated with losartan from 8 weeks of age and followed by serial echocardiography until they were killed at 9 months of age (FIG. 2C). Although there was a trend for increased aortic root growth in AT2KO:Fbn1^{C1039G/+} mice compared with Fbn1^{C1039G/+} littermates (P=0.06), the decrease in aortic root growth seen in AT2KO:Fbn1^{C1039G/+} mice treated with losartan was only 40% of that seen in Fbn1^{C1039G/+} animals that expressed AT2 (P<0.001), despite there being no difference in blood pressure between the groups (FIGS. 2C and 8). The modest reduction in aortic root growth seen in losartan-treated AT2KO:Fbn1^{C1039G/+} mice was comparable to that previously observed with propranolol, and it may be similarly attributable to a decline in blood pressure rather than a modulation of cytokine signaling (J. P. Habashi et al., Science 312, 117 (2006)).

Together, these experiments indicates that AT2 signaling protectively modifies MFS and that the therapeutic effect of ACEi likely relates to AT1 receptor blockade or antihypertensive effects. In addition, selective AT1 antagonism with the ARB losartan is beneficial in Fbn1^{C1039G/+} mice and that AT2 signaling is needed to achieve the full potential of ARBs.

### Example 4

### Protection by AT2 Receptor Signaling is Mediated by the Noncanonical ERK1/2 Signaling Cascade

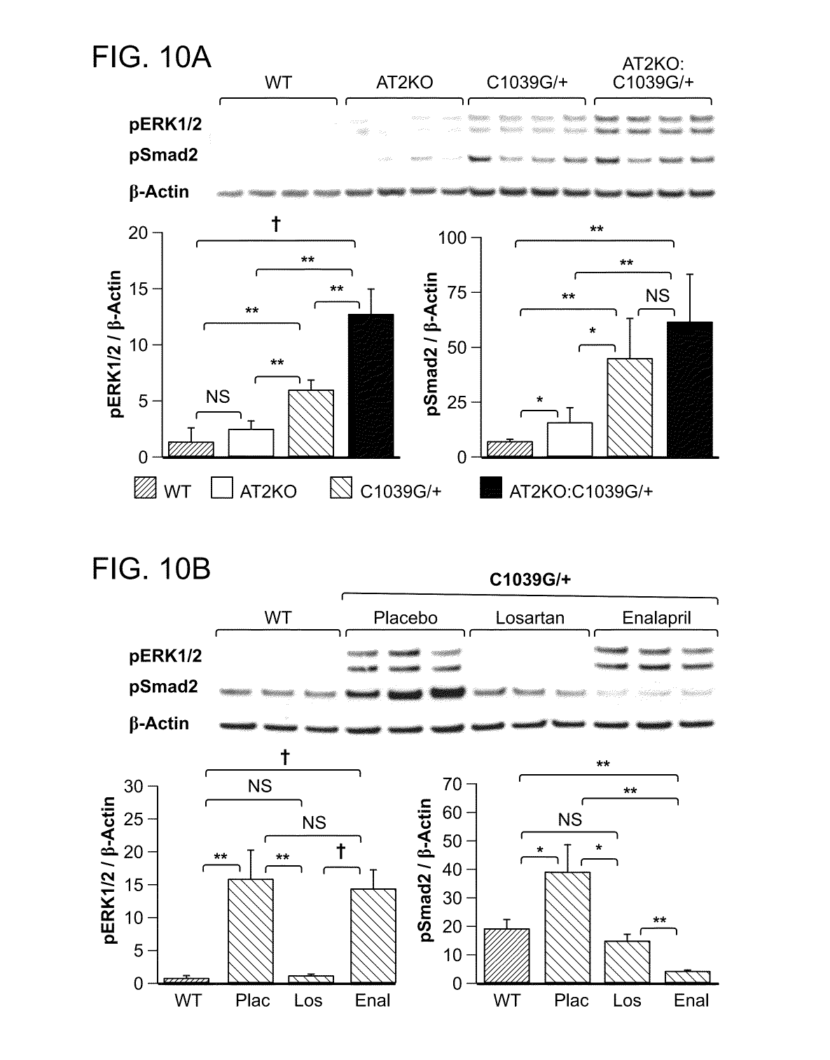

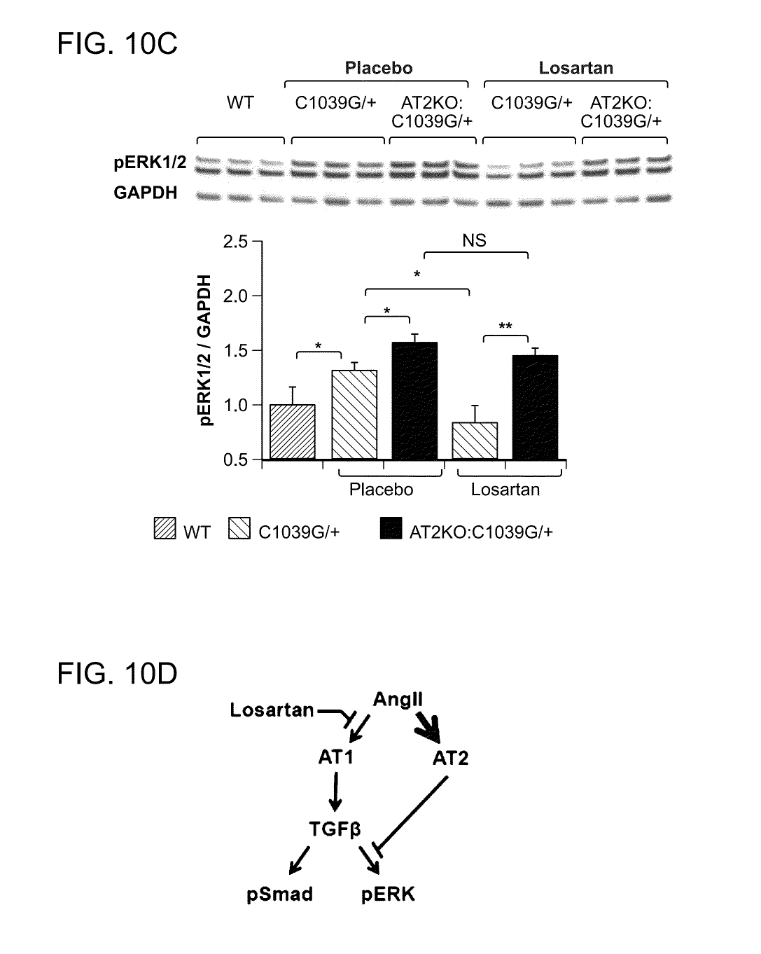

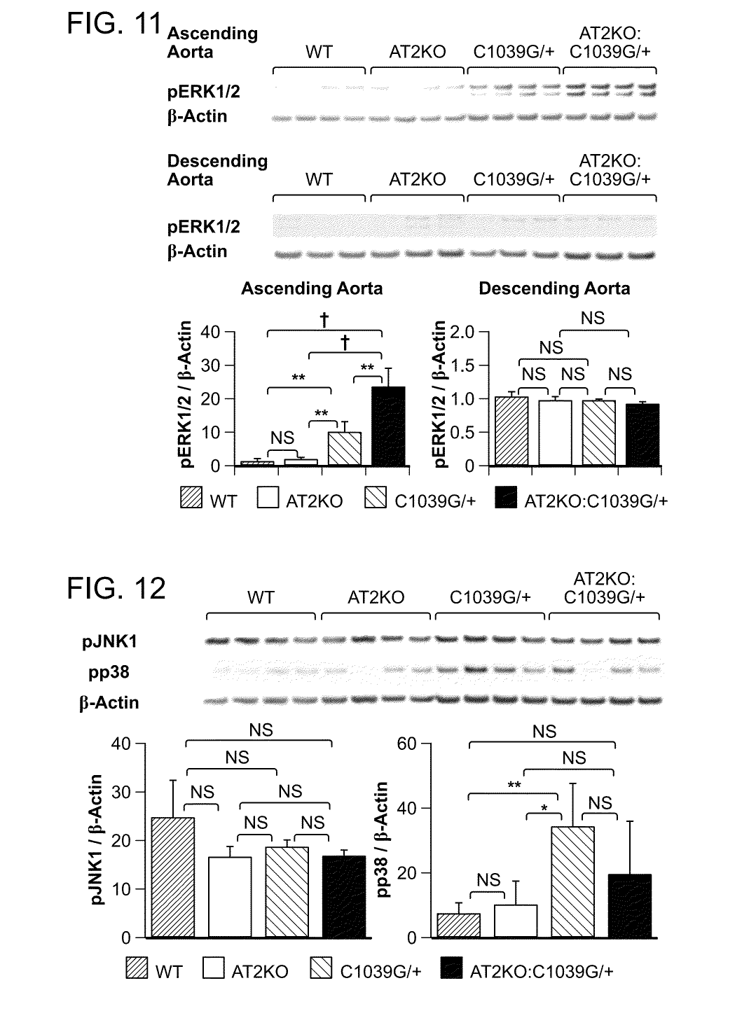

Both the canonical (Smad-dependent) and non-canonical (MAPK, predominantly ERK1/2 but also JNK in some experimental contexts) TGFβ signaling cascades are activated in Fbn1^{C1039G/+} mice in a TGFβ- and AT1 receptor—dependent manner (T. Holm et al., Science 332, 358 (2011)). To investigate the mechanism of protection by AT2 receptor signaling, the status of both canonical and noncanonical TGFβ signaling in Fbn1^{C1039G/+} mice lacking the AT2 receptor or in response to losartan or enalapril treatment was monitored. Western blot analysis showed that Smad2 activation was significantly greater in the aortic root and proximal ascending aorta of Fbn1^{C1039G/+} mice compared with WT controls (P<0.01) but that there was no significant difference between AT2KO:Fbn1^{C1039G/+} and Fbn1^{C1039G/+} mice (P=0.30). In contrast, ERK 1/2 activation was significantly greater in Fbn1^{C1039G/+} mice compared with WT litter-mates (P<0.01) and was further increased in AT2KO:Fbn1^{C1039G/+} mice compared with Fbn1^{C1039G/+} (P<0.01), AT2KO (P<0.01), and WT littermates (P<0.001) (FIG. 10A). The difference in ERK1/2 activation was specific to the aortic root and proximal ascending aorta, the areas most predisposed to aneurysm formation in MFS, as there was no significant difference in the descending thoracic aortas of the same animals (FIG. 11). No significant differences in JNK1 or p38 activation were observed (FIG. 12).

### Example 5

### The Biochemical Status of the Noncanonical ERK1/2 TGFβ Signaling Cascade Correlates with the Therapeutic Effects of ARBs and ACEi

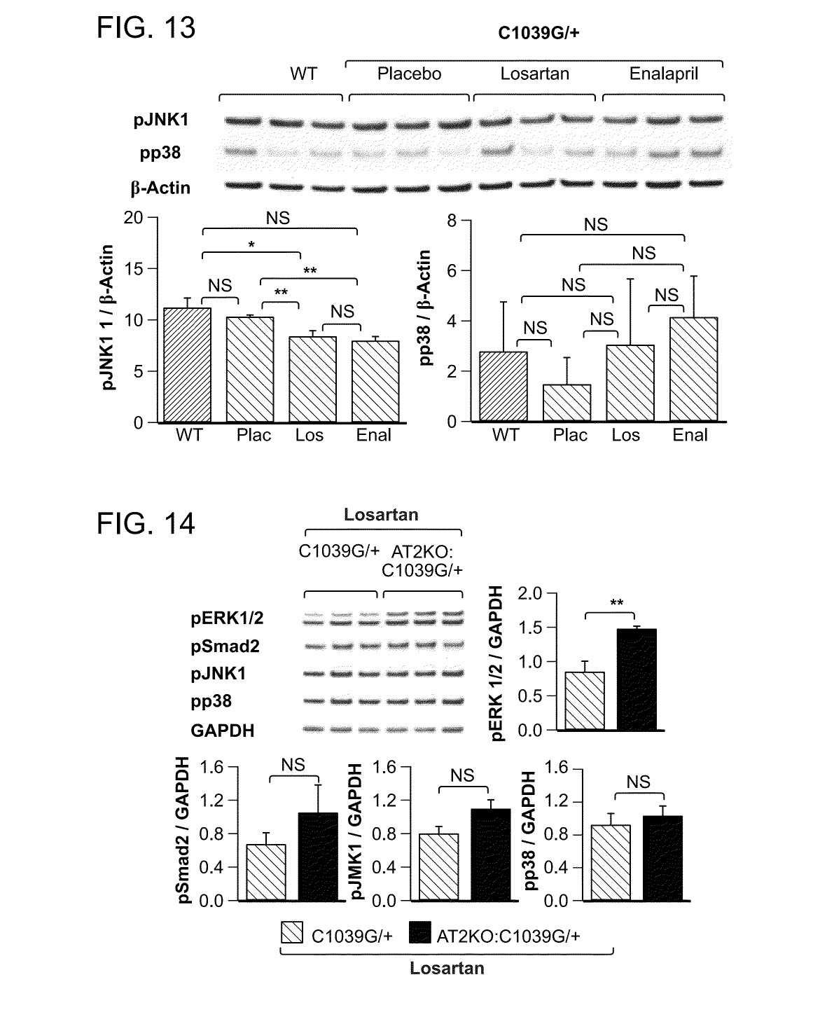

In the comparison of ARBs versus ACEi, Smad2 activation was significantly greater in Fbn1^{C1039G/+} mice compared with WT controls (P<0.05), and losartan treatment significantly decreased Smad2 activation in Fbn1^{C1039G/+} mice (P<0.05) to levels indistinguishable from WT (P=0.31) (FIG. 10B). Enalapril reduced Smad2 activation in Fbn1^{C1039G/+} mice significantly more than losartan (P<0.01), a finding that did not parallel the therapeutic effects of these agents (FIG. 2B). ERK1/2 activation was significantly greater in Fbn1^{C1039G/+} mice compared with WT controls (P<0.01), and treatment with losartan reduced it to WT levels (P=0.80). In contrast, enalapril treatment had significantly less effect on ERK1/2 activation than losartan (P<0.001); in fact, it was no more effective than placebo (P=0.50). JNK1 and p38 activation was similar in Fbn1^{C1039G/+} and WT mice; both losartan and enalapril caused a modest reduction in JNK1 activation (P<0.01), but neither had any effect on p38 activation (FIG. 13). Thus, the biochemical status of the noncanonical ERK1/2, but not the canonical Smad, TGFβ signaling cascade correlated with the therapeutic effects of these agents. In keeping with this finding, losartan had a reduced ability to lower ERK1/2 activation in Fbn1^{C1039G/+} mice lacking the AT2 receptor (P<0.01) (FIG. 10C). By contrast, there was no significant difference in Smad2, JNK1, or p38 activation in losartan-treated Fbn1^{C1039G/+} mice that did or did not express AT2 (FIG. 14).

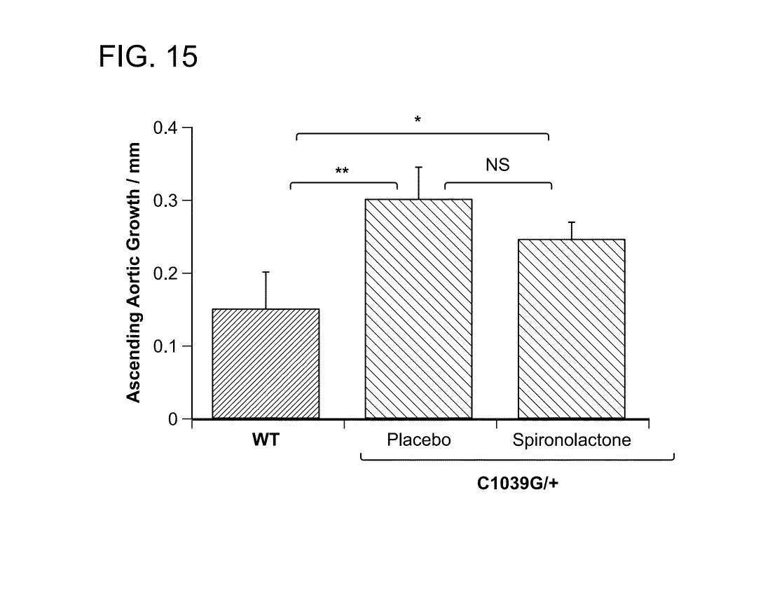

To assess for a contribution of other components of the renin-angiotensin-aldosterone system, we treated Fbn1^{C1039G/+} mice with the aldosterone receptor antagonist spironolactone (S. Sakurabayashi-Kitade et al., Atherosclerosis 206, 54 (2009)). We found no significant inhibition of aortic root growth over 7 months time (P=0.23) (FIG. 15).

In sum, dual blockade of AT1 receptor—mediated ERK1/2 activation and AT2 receptor—mediated ERK1/2 inhibition, as occurs either with the use of ACEi in Fbn1^{C1039G/+} mice or the use of losartan in AT2KO:Fbn1^{C1039G/+} mice, results in no net change in ERK1/2 activation status and adds a very modest therapeutic benefit. By contrast, losartan reduces ERK1/2 phosphorylation through a combination of both inhibiting AT1 receptor—mediated ERK activation and by shunting AngII signaling through the AT2 receptor. This indicates that, in the presence of AT1 receptor blockade, ongoing AT2 receptor signaling is required for the attenuation of ERK phosphorylation and that enalapril's lack of effect on ERK is attributable to the loss of AT2 receptor signaling potential with this agent (FIG. 10D). Given that the small reduction in aortic root growth in Fbn1^{C1039G/+} mice achieved by enalapril in this study was comparable to that achieved previously by propanolol (J. P. Habashi et al., Science 312, 117 (2006)), this indicates that its small beneficial effect may well have been mediated by blood pressure reduction. Although the concordant effects of prior manipulations and therapies on canonical and noncanonical TGFβ signaling made it impossible to dissect their relative contributions, the differential effects of enalapril treatment indicate that TGFβ-mediated ERK1/2 activation is the predominant driver of aneurysm progression in MFS. In light of this, analysis of ERK1/2 activation status will allow for the optimization of dosing regimens for losartan or other ARBs in ongoing or future clinical trials in people with MFS. Furthermore, the ERK1/2 signaling cascade represents new therapeutic targets in the treatment of aortic aneurysm disease.

Examples 1 to 5 were Carried Out Using the Following Materials and Methods.

### Mice

All mice were cared for under strict compliance with the Animal Care and Use Committee of the Johns Hopkins University School of Medicine. The Fbn1^{C1039G/+} and AT2KO lines were maintained n a pure C57BL/6 background (backcrossed for greater than 9 generations), allowing for valid comparisons. In order to further accommodate the potential for temporal- or background-specified variation, all comparisons were made between contemporary littermates when possible. The AT21C0 mice were obtained as a generous gift from Dr. Inagami (T. Ichiki et al., Nature 377, 748 (1995)). The Agtr2 gene resides on the X chromosome and therefore we used either male mice carrying the mutated allele (who are hemizygous) or homozygous females, both of which have been previously shown to be functionally null for the AT2 receptor (T. Ichiki et al., Nature 377, 748 (1995)). Mice were sacrificed with an inhalation overdose of halothane (Sigma-Aldrich, St. Louis). Mice underwent immediate laparotomy, descending abdominal aortic transection, and PBS (pH 7.4) was infused through the right and left ventricles to flush out the blood. Mice that were analyzed for Western Blot analysis had their proximal ascending aortas (root to right brachiocephalic trunk) immediately dissected out, flash frozen in liquid nitrogen and stored at −80d until further processing. Mice that were analyzed for aortic histology had latex injected under low pressure into the left ventricular apex until it was visible in the descending abdominal aorta. Mice that were analyzed for lung histology had their trachea intubated with a 20-gauge blunted needle, and 0.5% agar was infused under a low and constant pressure to gradually inflate the lungs. The trachea was then tied-off using vicryl and the needle was removed. Mice were fixed for 24 hours in 10% buffered formalin, after which the heart, aorta and lungs were removed and stored in 70% ethanol.

### Delivery of Medication

Mice were started on medication at 8 weeks of age and continued for 7 months. Losartan was dissolved in drinking water and filtered to reach a concentration of 0.6 g/L, giving an estimated daily dose of 40-60 mg/kg/day. Enalapril was dissolved in drinking water and filtered to reach a final concentration of 0.15 g/L, giving an estimated daily dose of 10-15 mg/kg/day. These doses were chosen to achieve a comparable hemodynamic effect, as previously described (R. D. Patten et al., Clin. Sci 104, 109 (2003)). Spironolactone was dissolved in drinking water and filtered to give an estimated dose of 20 mg/kg/day, a dose previously shown to have in-vivo phenotypic benefit in cardiovascular disease states (S. Sakurabayashi-Kitade et al., Atherosclerosis 206, 54 (2009)). Placebo-treated animals received regular drinking water. Blood pressures were analyzed by taking 20 tail cuff blood pressures per day over 5 days in each mouse to habituate the mice to the tail cuff pressure system, and the blood pressures obtained on the last day were averaged. At least 4 mice for each treatment group were analyzed.

### Echocardiography

Nair hair removal cream was used on all mice the day prior to echocardiograms. All echocardiograms were performed on awake, unsedated mice using the Visualsonics Vevo 660 V1.3.6 imaging system and a 30 MHz transducer. Mice were imaged at baseline, 1 month after treatment and then every two months until the time of sacrifice. The aorta was imaged using a parasternal long axis view. Three separate measurements of the maximal internal dimension at the sinus of Valsalva and proximal ascending aorta were made from distinct captured images and averaged. All imaging and measurements were performed by a cardiologist who was blinded to genotype and treatment arm.

### Antibodies and Western Blot Analysis

Mouse aortic root and ascending aortas (aortic root excluding the aortic valve to origin of right brachiocephalic trunk) were harvested, snap-frozen in liquid nitrogen and stored at −80° C. until processed. Protein was extracted using the reagents and protocol from a Total Protein Extraction Kit containing protease inhibitor and Protein Phosphatase Inhibitor Cocktail (Millipore, MA). Aortas were homogenized using a pellet pestle motor (Kimble-Kontes, NJ) as per the extraction kit protocol. Homogenates were dissolved in sample buffer, run on a NuPAGE Novex 4-12% Bis-Tris Gel (Invitrogen, CA), and transferred to nitrocellulose membranes using the iBlot transfer system (Invitrogen, CA). Membranes were washed in phosphate-buffered saline (PBS) and blocked for 1 hr at room temperature with 5% instant non-fat dry milk dissolved in PBS containing 1% Tween-20 (Sigma, Mo.) (PBS-T). Equal protein loading of samples was determined by a protein assay (BioRad, CA), and confirmed by probing with antibodies against β-Actin or GAPDH (Sigma, Mo.). Membranes were probed overnight at 4° C. with primary antibodies against pERK1/2, pJNK1/2 (Santa Cruz, Calif.), pSmad2 and pp 38 (Cell Signaling, CA) dissolved in PBS-T containing 5% milk. Blots were then washed in PBS-T and probed with HRP-conjugated anti-rabbit or anti-mouse secondary antibodies (GE Healthcare, UK) dissolved in PBS-T containing 5% milk at room temperature. Blots were then washed in PBS-T, developed using SuperSignalWest HRP substrate (Pierce Scientific, IL), exposed to BioMax Scientific Imaging Film (Sigma, Mo.) and quantified using Imaged analysis software (NIH, MD).

### Histological and Morphometric Analysis

Latex-infused ascending aortas were transected just above the level of the aortic valve, and 2-3 mm transverse sections were mounted in 4% agar prior to paraffin fixation. Five micrometer aortic sections underwent Verhoeff-van Giesen (VVG) staining and were imaged at 40× magnification, using a Nikon Eclipse E400 microscope. Wall thickness of the aortic media was measured by a single blinded observer at 16 different locations around the most proximal ascending aortic ring and averaged. Wall architecture of 4 representative sections for each mouse was assessed by the same 3 blinded observers and graded on an arbitrary scale of 1 (indicating no breaks in the elastic fiber) to 5 (indicating diffuse fragmentation), and the results were averaged. Elastic fiber content was quantified in four separate representative images of each section of the most proximal ascending aorta by a single blinded observer, using NIS Elements Advanced Research (Nikon, Japan). The aortic media and the elastic fibers were individually outlined and their areas calculated. The respective areas were averaged from all the images of a given aortic section and the ratio of elastic fiber content to total aortic media was determined. Individual lobes of the lungs were mounted in 4% agar and fixed in paraffin. Five micrometer lung sections underwent hematoxylin and eosin staining and were imaged at 10× magnification, using a Nikon Eclipse E400 microscope. Five fields were analyzed for each lobe of each lung by a single blinded observer, and a mean linear intercept was calculated using a previously described method (J. P. Habashi et al., Science 312, 117 (2006); and E. R. Neptune et al., Nat. Genet. 33, 407 (2003)).

### Statistical Analysis

All values are expressed as mean±2 standard errors of the mean (SEM). One way ANOVA was used to evaluate significance between groups with a p-value of <0.05 considered statistically significant.

### Example 6

### Fbn1^{C1039G/+} Mice Treated with Either TGFβNAb or Losartan Show a Significant Reduction in ERK1/2 Activation

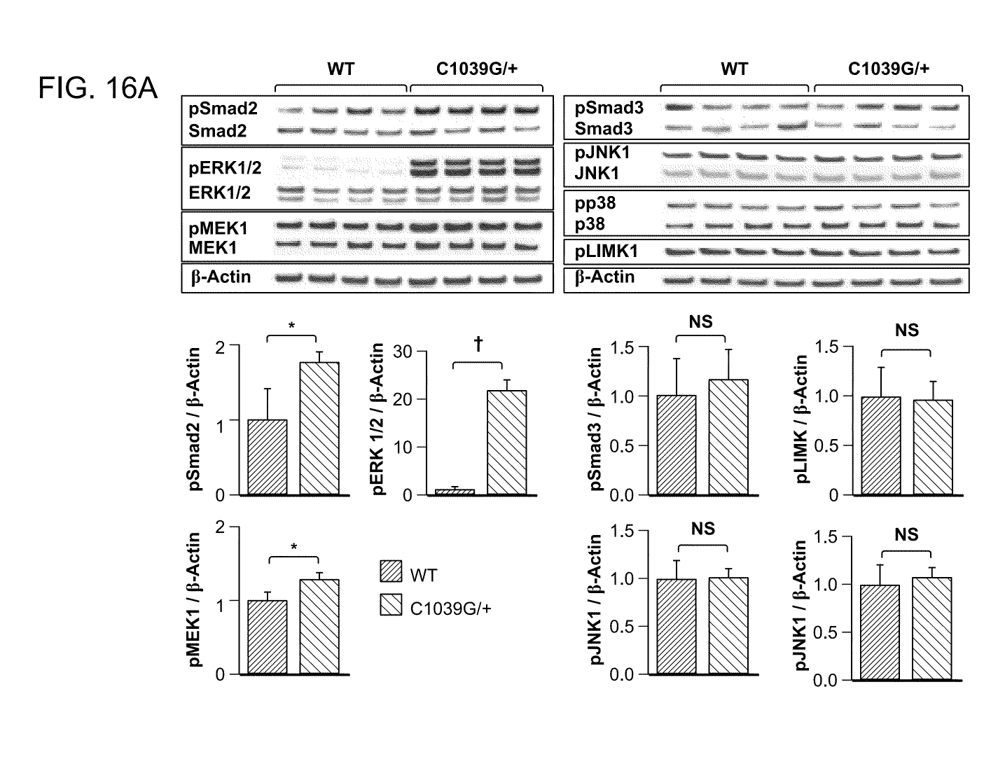

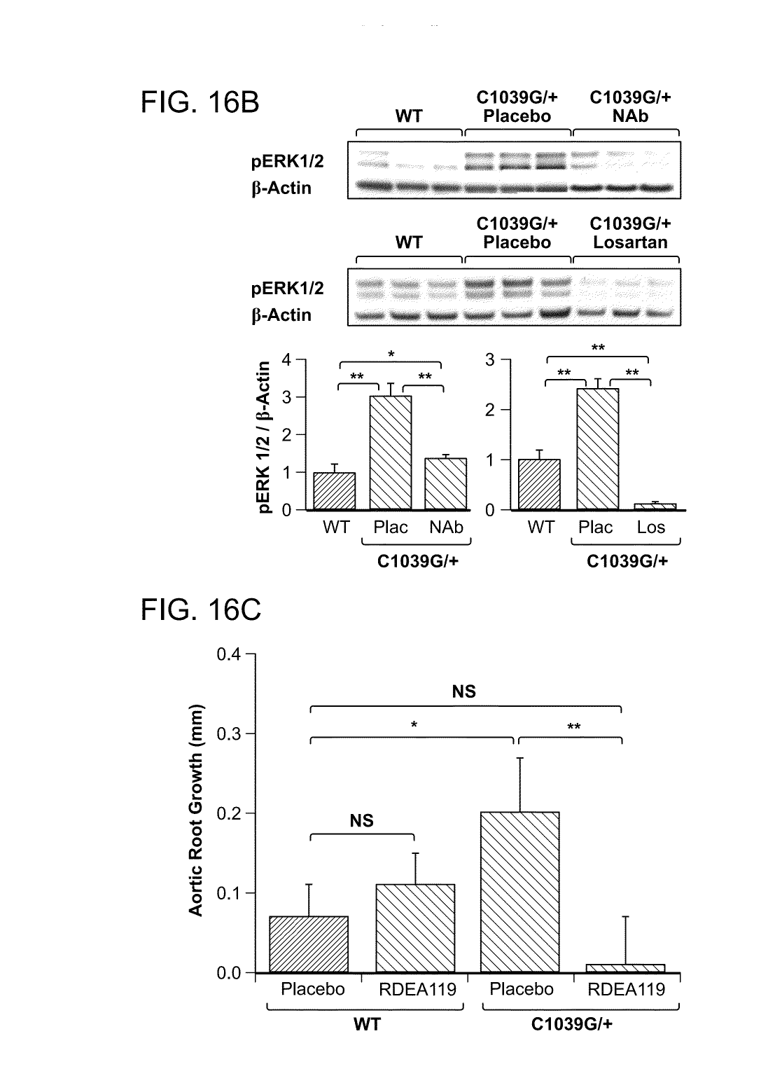

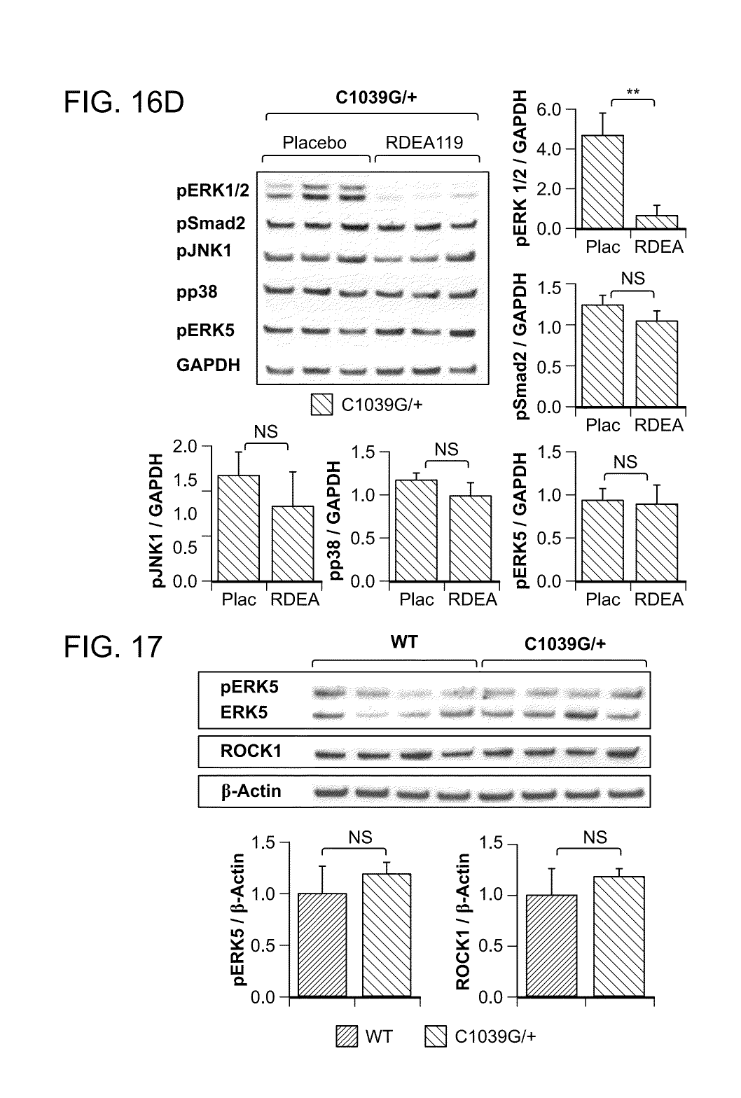

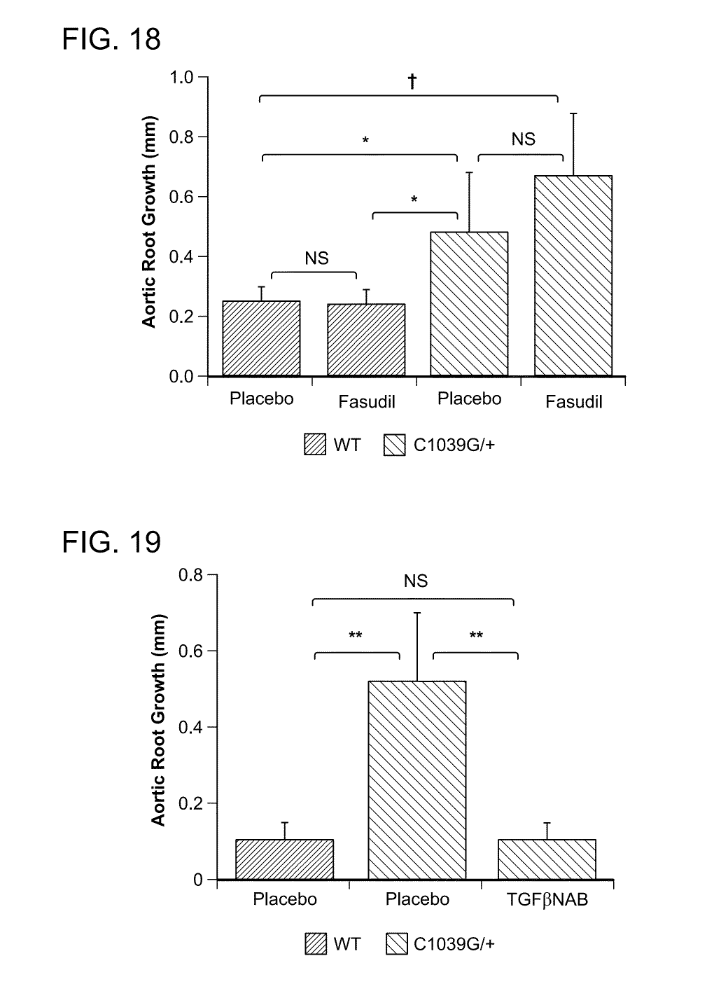

Western blot analysis was performed on the proximal ascending aorta of 12-month-old mice heterozygous for a missense mutation in Fbn 1 (Fbn1^{C1039G/+}), a validated animal model of MFS (D. P. Judge et al., J. Clin. Invest. 114, 172 (2004)). Compared with wild-type (WT) littermates, Fbn1^{C1039G/+} mice showed a significant increase in activation of Smad2, ERK1/2, and MAPK kinase 1 (MEK1), the upstream activator of ERK1/2 (P<0.05, P<0.001, and P<0.05, respectively) (FIG. 16A). In contrast, there was no difference in the activation of Smad3; JNK1; p38; ERK5; Rho-associated coiled-coil containing protein kinase-1 (ROCK1); or LIMK1, a downstream target of ROCK1 (FIGS. 16A and 17). Furthermore, an in vivo trial of fasudil, a well-established inhibitor of the RhoA/ROCK pathway failed to attenuate aortic root growth in Fbn1^{C1039G/+} mice (FIG. 18).

Because TGFβNAb and losartan attenuate aortic root growth in Fbn1^{C1039G/+} mice (FIG. 19) (J. P. Habashi et al., Science 312, 117 (2006)), if Smad2 or ERK1/2 are important mediators of aortic disease in MFS, one would expect their activation to be reduced by these agents. Prior work demonstrated that Smad2 activation is decreased by both therapies (J. P. Habashi et al., Science 312, 117 (2006)). As shown in FIG. 16B, compared with placebo-treated littermates, Fbn1^{C1039G/+} mice treated with either TGFβNAb or losartan also show a significant reduction in ERK1/2 activation (P<0.01 for both).

### Example 7

### Inhibition of the ERK1/2 Signaling Cascade Reduced Aortic Root Growth in MFS Mice

To confirm that ERK1/2 is a driver, rather than simply a marker, of aortic aneurysm progression, 2-month-old Fbn1^{C1039G/+} mice were treated for 2 months with the selective MEK1/2 inhibitor RDEA119 (C. Iverson et al., Cancer Res. 69, 6839 (2009)). Aortic root size was measured by echocardiography at 2 months (baseline before treatment) and 4 months of age (FIG. 16C). Aortic root growth was significantly greater in placebo-treated Fbn1^{C1039G/+} mice, compared with WT littermates (P<0.05). Aortic root growth in RDEA119-treated Fbn1^{C1039G/+} mice was significantly less than that of placebo-treated Fbn1^{C1039G/+} littermates (P<0.01) and indistinguishable from that observed in WT mice (P=0.15). RDEA119 therapy had no significant effect in WT mice (P=0.24), which illustrated that inhibition of ERK1/2 activation specifically targets MFS-associated pathological aortic root growth, while still allowing for normal physiological growth.

The specificity of RDEA119 was confirmed by Western blot analysis of the proximal ascending aorta. Compared with placebo-treated Fbn1^{C1039G/+} littermates, RDEA119-treated Fbn1^{C1039G/+} mice showed a significant reduction in ERK1/2 activation (P<0.01), whereas Smad2, JNK1, p38 and ERK5 activation was unchanged (FIG. 16D). This result also shows that Smad2 activation in Fbn1^{C1039G/+} mice is not ERK-dependent. Together, these data indicate that TGFβ-driven ERK1/2 activation contributes to aortic aneurysm progression in MFS mice and that antagonism of this pathway will be therapeutically useful.

### Example 8

### Inhibition of JNK1 Ameliorated Aortic Root Growth in MFS Mice

To determine whether canonical signaling contributes to aortic disease progression in MFS, we introduced haploinsufficiency for Smad4, a critical mediator of canonical TGFβ signaling, into our MFS mouse model. We bred Fbn1^{C1039G/+} mice to mice harboring a deletion of exon 8 of the Smad4 gene. Homozygosity for this Smad4 allele (S4^{-/-}) results in the death of embryos (X. Xu et al., Oncogene 19, 1868 (2000)). In contrast, haplo insufficient mice (S4^{+/-}) are fertile, have normal life spans, and show clinically relevant attenuation of Smad-dependent signaling in several tissues, including the stomach, breast, and intestine (X. Xu et al., Oncogene 19, 1868 (2000)).

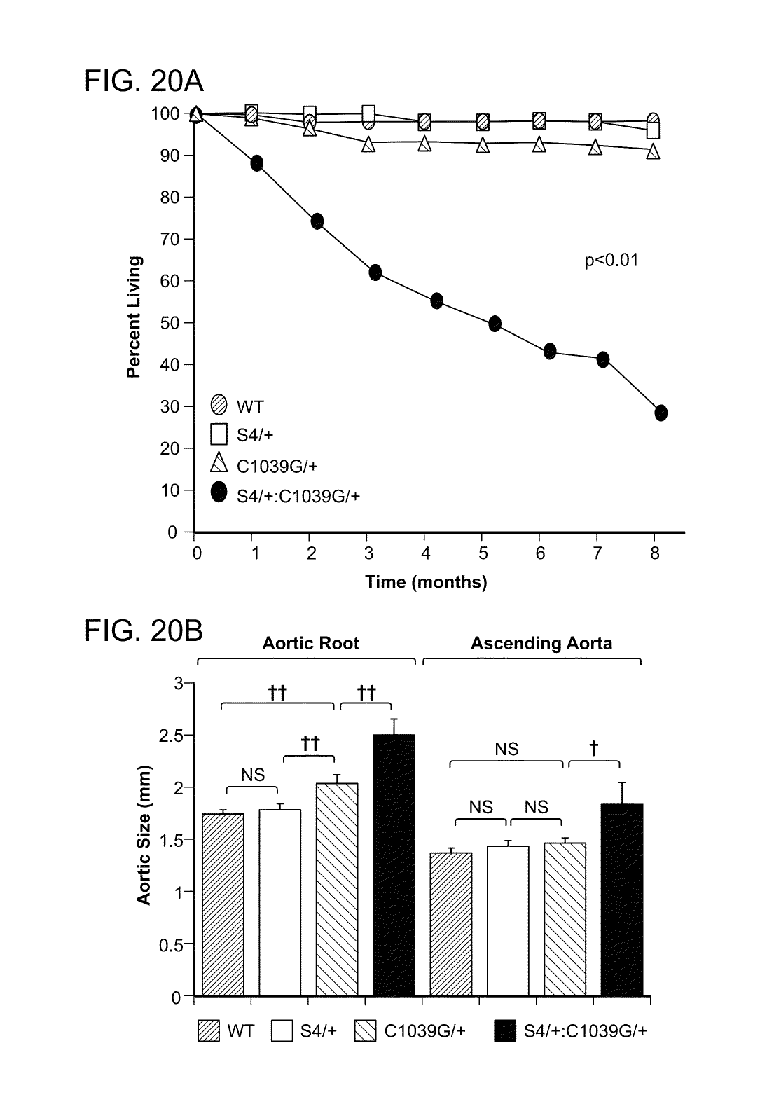

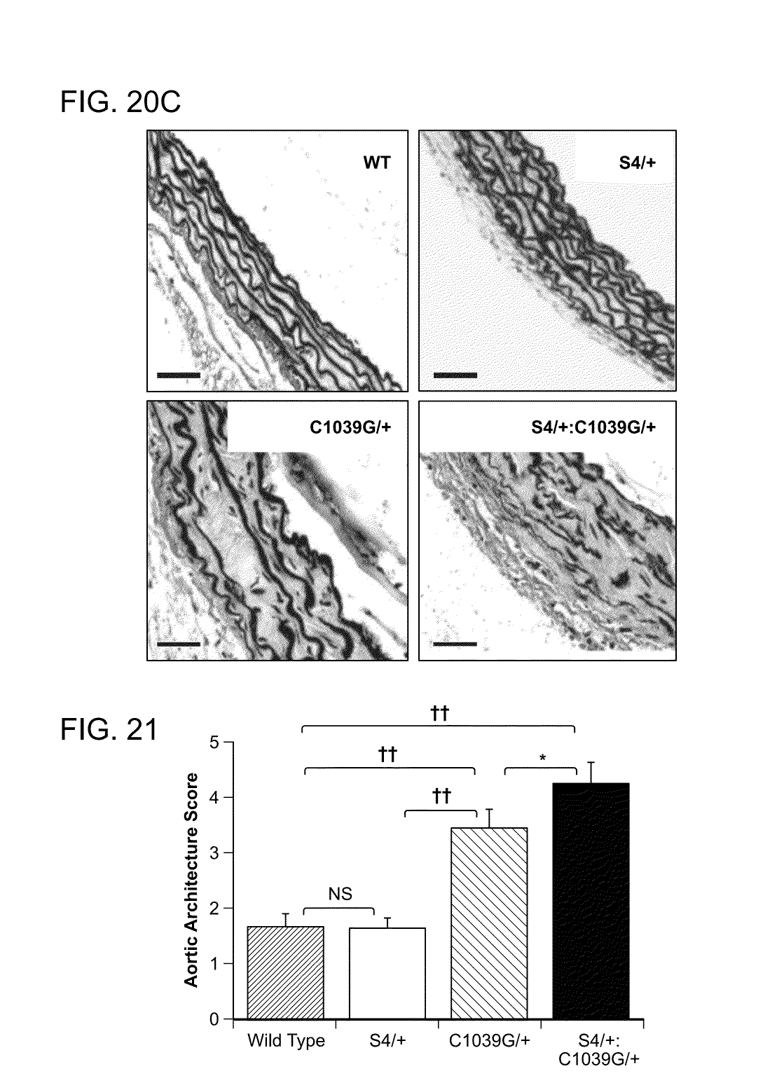

The Fbn1^{C1039G/+} MFS mouse model shows progressive aortic root dilatation, but does not typically progress to aortic dissection or premature death. Whereas almost all WT, S4^{+/-0} and Fbn1^{C1039G/+} mice survived to 8 months of age, S4^{+/-}:Fbn1^{C1039G/+} mice died prematurely. This was first evident by 1 month of age; by 3 months 40% had died, and by 8 months 70% had died (FIG. 20A). Necropsy of these animals revealed hemothorax and hemopericardium in all cases, indicative of proximal aortic rupture; there was no evidence of aortic rupture in any WT, S4^{+/-}, or Fbn1^{C1039G/+} mice.

Echocardiography at 3 months of age revealed significant enlargement of both the aortic root and the ascending aorta in S4^{+/-}:Fbn1^{C1039G/+} mice, compared with Fbn1^{C1039G/+} littermates (P<0.0001 and P<0.001, respectively) (FIG. 20B). These data provide a conservative estimate of the effect of Smad4 haploinsufficiency, because premature deaths in the S4^{+/-}:Fbn1^{C1039G/+} cohort effectively eliminated more severe cases from the analysis. No difference in aortic root or ascending aortic size was observed between WT and S4^{+/-} mice (P=0.20 and P=0.20, respectively) (FIG. 20B), which indicated that the deleterious effect of Smad4 haploinsufficiency was limited to MFS mice.

After death of the mice, we performed Verhoeff-Van Gieson (VVG) staining of the proximal ascending aorta to assess whether there were any abnormalities in aortic architecture (FIG. 20C). Compared with WT littermates, Fbn1^{C1039G/+} mice showed increased aortic medial thickening, elastic fiber fragmentation, and elastic fiber disarray, collectively quantified as an aortic architecture score (P<0.0001) (FIG. 21). S4^{+/-}:Fbn1^{C1039G/+} mice showed an exaggeration of these pathologic changes (P<0.05). By contrast, there was no significant difference between WT and S4^{+/-} mice (P=0.94). The histological changes in the aorta therefore paralleled the echocardiography findings and support the conclusion that Smad4 haploinsufficiency exacerbates aortic disease in MFS mice.

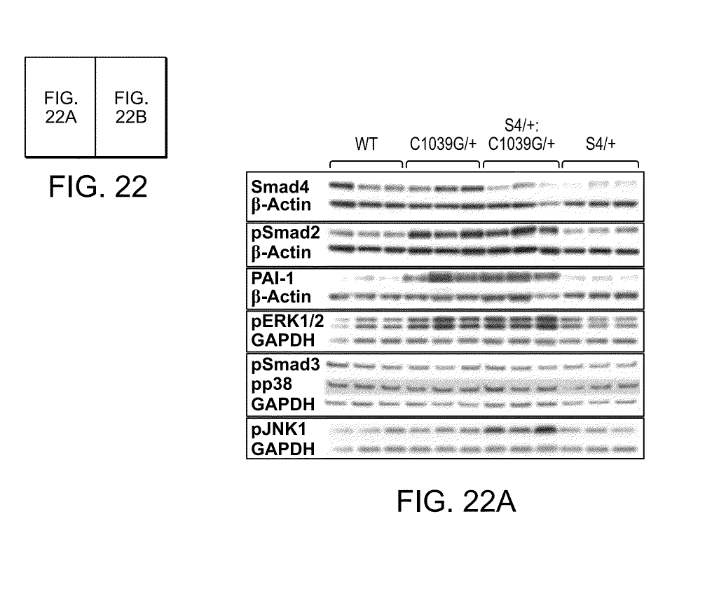

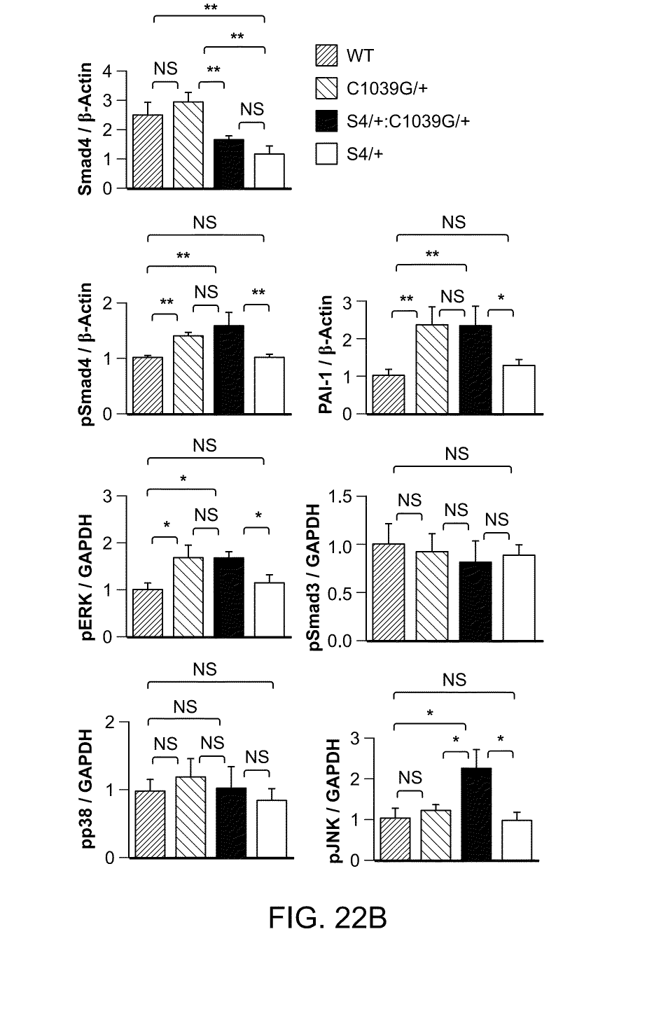

Using Western blot analysis, we evaluated the effect of Smad4 haploinsufficiency on canonical and noncanonical TGFβ signaling in the proximal ascending aorta (FIG. 22). As anticipated, S4^{+/-} and S4^{+/-}:Fbn1^{C1039G/+} mice showed a roughly 50% reduction in expression of Smad4 protein, compared with WT and Fbn1^{C1039G/+} mice. Compared with WT animals, Fbn1^{C1039G/+} mice showed significantly greater activation of Smad2 and ERK1/2 (P<0.01 and P<0.05, respectively) and a significant increase in expression of the Smad2-responsive gene product PAI-1 (P<0.01). However, there was no further increase in Smad2 activation, ERK1/2 activation, or PAI-1 expression in S4^{+/-}:Fbn1^{C1039G/+} mice (P=0.35, P=0.90, and P=0.85, respectively). This indicates that Smad4 haploinsufficiency did not attenuate Smad-dependent signaling and that increased Smad2 or ERK1/2 activation could not be invoked as the cause of the aortic disease exacerbation seen in S4^{+/-}:Fbn1^{C1039G/+} mice.

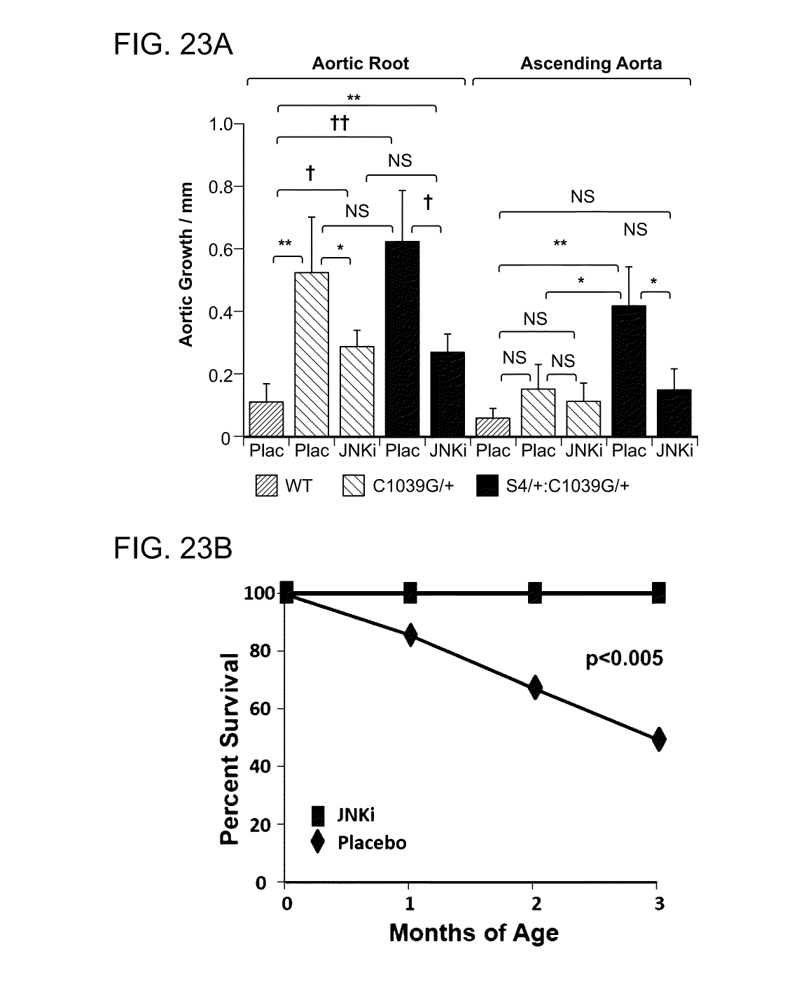

We next assessed whether other TGFβ-dependent canonical or noncanonical pathways could account for these changes (FIG. 22). There was no significant difference in Smad3 or p38 activation in WT, Fbn1^{C1039G/+}, or S4^{+/-}:Fbn1^{C1039G/+}. Although there was no significant difference in JNK1 activation between Fbn1^{C1039G/+} and WT mice, S4^{+/-}:Fbn1^{C1039G/+} mice demonstrated unique activation of JNK1 (P<0.05). We therefore treated a cohort of S4^{+/-}:Fbn1^{C1039G/+} mice with SP600125, a selective JNK inhibitor (B. L. Bennett et al., Proc. Natl. Acad. Sci. U.S.A. 98, 13681 (2001)). SP600125 treatment led to a significant reduction in both aortic root and ascending aortic growth in S4^{+/-}:Fbn1^{C1039G/+} mice, compared with placebo-treated littermates (P<0.001 and P<0.05, respectively) (FIG. 23A). Furthermore, SP600125 treatment prevented the premature death due to aortic dissection seen in these animals. At 3 months of age, 50% of placebo-treated S4^{+/-}:Fbn1^{C1039G/+} mice had died from aortic dissection, whereas all of the SP600125 treated S4^{+/-}:Fbn1^{C1039G/+} mice were still alive (FIG. 23B).

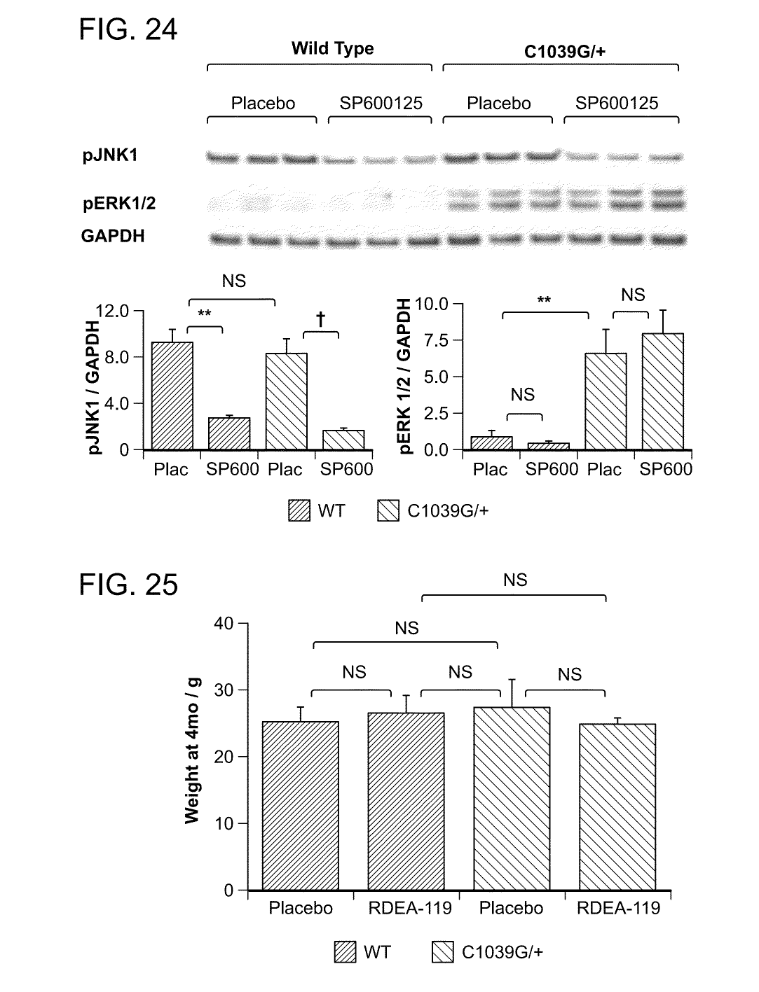

Although JNK1 activation is not increased in the aortas of Fbn1^{C1039G/+} mice, SP600125 treatment ameliorated their aortic root growth, compared with placebo-treated Fbn1^{C1039G/+} littermates (P<0.05) (FIG. 23A). This correlated with a reduction of JNK1 activation to levels below baseline, whereas ERK1/2 activation remained unaffected, in SP600125-treated Fbn1^{C1039G/+} animals (FIG. 24). This observation is consistent with prior work showing that SP600125 can ameliorate abdominal aortic aneurysm induced by the periaortic application of calcium chloride, in association with reduced JNK1 activation (K. Yoshimura et al., Nat. Med. 11, 1330 (2005)). These data indicate that both ERK1/2 and JNK1 can contribute to aortic disease in fibrillin-1-deficient mice; whether or not this relies upon downstream cross-talk between these signaling cascades remains to be determined. Taken together, these data further support the conclusion that noncanonical TGFβ signaling is a prominent determinant of aortic aneurysm progression in MFS mice. ERK activation was recently shown to occur in the aorta of a fibulin-4-deficient mouse model of cutis laxa with aneurysm, although a mechanistic link remains to be elucidated (I. Huang et al., Circ. Res. 106, 583 (2010)). ERK activation also appears to be central to the pathogenesis of cardiovascular disease in Noonan syndrome (T. Araki et al., Nat. Med. 10, 849 (2004); and T. Nakamura et al., J. Clin. Invest. 117, 2123 (2007)). Although aortic aneurysm has been described in this condition (J. M. Morgan, M. O. Coupe, M. Honey, G. A. Miller, Eur. Heart J. 10, 190 (1989); and R. Purnell, I. Williams, U. Von Oppell, A. Wood, Eur. J. Cardiothorac. Surg. 28, 346 (2005)), it is not highly penetrant, which suggests as yet undefined context specificity. It is also notable that the combination of aortic root and ascending aortic aneurysm seen in S4^{+/-}:Fbn1^{C1039G/+} mice is similar to that observed in individuals with wither Loeys-Dietz syndrome or bicuspid aortic valve and aneurysm. Both conditions are associated with increased TGFβ signaling in the aortic wall (B. L. Loeys et al., Nat. Genet. 37, 275 (2005); and D. Gomez et al., J. Pathol. 218, 131 (2009)), but the contribution of noncanonical TGFβ signaling cascades has not been revealed. In sum, this work defines a critical role for noncanonical TGFβ-dependent signaling in aneurysm pathogenesis in MFS mice. It also defines inhibition of ERK1/2 or JNK1 activation as therapeutic strategies for MFS and indicates that such therapies may find broader application. Finally, it focuses attention on noncanonical TGFβ signaling cascades in MFS-related conditions, where etiology or pathogenesis remains poorly understood.

Examples 6 to 8 were Carried Out Using the Following Materials and Methods

### Mice

All mice were cared for under strict compliance with the Animal Care and Use Committee of the Johns Hopkins University School of Medicine. The Smad4 haploinsufficient mice were a generous gift from Dr. Chuxia Deng (NIH/NIDDK, Bethesda). The Fbn1^{C1039G/+} line was maintained on a C57BL/6 background, allowing for valid comparisons. In order to further accommodate the potential for temporal- or background-specific variation in genetic or pharmacological manipulation experiments, all comparisons were made between contemporary littermates. Mice were checked daily for death and all mice found dead were immediately necropsied to assess for evidence of aortic dissection. Mice were killed with an inhalation overdose of halothane (Sigma-Aldrich, St. Louis). Mice underwent immediate laparotomy, descending abdominal aortic transection, and PBS (pH 7.4) infusion through the left ventricle to flush out the blood. For Western blot analysis, the proximal ascending aorta (root to right brachiocephalic trunk) was removed, flash frozen in liquid nitrogen and stored at −80° C. Following PBS infusion, mice analyzed for aortic histology had latex injected under low pressure into the left ventricular apex until it was visible in the descending abdominal aorta. The mice were then fixed for 24 hours in 10% buffered formalin, after which the heart and aorta were removed and stored in 70% ethanol.

### Medication

Mouse monoclonal TGFβNAb (1d11; R&D Systems, Minneapolis) was reconstituted in PBS and administered via intraperitoneal injection 3 times a week at a dose of 5 mg/kg. Treatment was initiated at 1 month of age and continued for 2 months. IgG (Zymed Laboratories Inc, San Francisco) was reconstituted in PBS, and administered at a dose of 10 mg/kg as a control. SP600125 (Sigma-Aldrich, St. Louis) was reconstituted in 10% DMSO dissolved in PBS, and administered twice daily by intraperitoneal injection, at a dose of 30 mg/kg. Treatment was initiated at 1 month of age and continued for 2 months. 10% DMSO dissolved in PBS was administered as a control. RDEA119 was reconstituted in 10% 2-hydroxypropyl-beta-cyclodextrin (Sigma-Aldrich, St. Louis) dissolved in PBS, and administered twice daily by oral gavage at a dose of 25 mg/kg. Treatment was initiated at 2 months of age and continued for 2 months. 10% 2-hydroxypropyl-beta-cyclodextrin dissolved in PBS was administered as a control. Fasudil was dissolved in drinking water, and administered at a dose of 1 mg/kg body weight per day. Treatment was initiated at 2 months of age and continued for 4 months. Drinking water was administered as a control.

### Echocardiography

Nair hair removal cream was used on all mice the day prior to echocardiography. All echocardiograms were performed on awake, unsedated mice using the Visualsonics Vevo 660 V1.3.6 imaging system and a 30 MHz transducer. The aorta was imaged using a parasternal long axis view. Three separate measurements of the maximal internal systolic dimension at the sinus of Valsalva and proximal ascending aorta were made, and a mean was calculated. All imaging and measurements were performed by a cardiologist who was blinded to genotype and treatment arm. In the TGFβNAb and SP600125 trials, mice were imaged at 1 month (baseline) and 3 months of age, after which they were killed. In the RDEA119 trial, mice were imaged at 2 months (baseline) and 4 months of age, after which they were killed. In the fasudil trial, mice were imaged at 2 months (baseline) and 6 months of age, after which they were killed. Smad4 haploinsufficient mice were imaged at 1 month, and then every 2 months thereafter, until death or sacrifice.

### Western Blot Analysis

Protein was extracted using the reagents and protocol from a Total Protein Extraction Kit, in conjunction with a Protein Phosphatase Inhibitor Cocktail (Millipore, MA). Aortas were homogenized using a pellet pestle motor (Kimble-Kontes, NJ) as per the extraction kit protocol. Homogenates were dissolved in sample buffer, run on a NuPAGE Bis-Tris Gel (Invitrogen, CA), and transferred to nitrocellulose membranes using the iBlot transfer system (Invitrogen, CA). Membranes were washed in PBS and blocked for 1 hour at room temperature with 5% instant non-fat dry milk, dissolved in PBS containing 1% Tween-20 (Sigma, Mo.) (PBS-T). Equal protein loading of samples was determined by a protein assay (BioRad, CA) and confirmed by probing with antibodies against β-Actin or GAPDH (Sigma, Mo.). Membranes were probed overnight at 4° C. with primary antibodies from Santa Cruz, Calif. (pERK1/2, pJNK1/2) and Cell Signaling, CA (pSmad2, Smad2, ERK1/2, JNK1, pp 38, p38, pMEK1, MEK1, pERKS, ERK5, ROCK1, pLIMK1, Smad4, pSmad2, PAI-1, pSmad3 and Smad3) dissolved in PBS-T containing 5% milk. Blots were then washed in PBS-T, and probed for 1 hour at room temperature with HRP-conjugated secondary antibodies (GE Healthcare, UK) dissolved in PBS-T containing 5% milk. Blots were then washed in PBS-T, developed using SuperSignalWest HRP substrate (Pierce Scientific, IL), exposed using BioMax Scientific Imaging Film (Sigma, Mo.) and quantified using ImageJ analysis software (NIH, MD).

### Histological Analysis

Latex-infused ascending aortas were transected just above the level of the aortic valve, and 2- to 3-mm transverse segments were mounted in 4% agar. These were then paraffin embedded and sectioned. Sections underwent Verhoeff-van Giesen (VVG) staining and were imaged at 40× magnification, using a Nikon Eclipse E400 microscope. Four representative VVG images of each mouse aorta were assessed by 3 blinded observers and graded on a scale of 1 (indicating no elastic fiber breaks) to 5 (indicating extensive elastic fiber fragmentation). An aortic wall architecture score was calculated by averaging the results of the 3 blinded observers. Sections also underwent trichrome staining to assess the degree of collagen deposition in the aortas of these mice.

### Statistical Analysis

All values are expressed as means±2 standard errors of the mean (2 SEM). Student t tests were used to evaluate significance between groups, with a p-value of <0.05 considered statistically significant.

To evaluate the effect of RhoA/ROCK pathway inhibition on aortic root growth, WT and Fbn1^{C1039G/+} mice were treated with fasudil at a dose (1 mg/kg) previously shown to rescue ROCK-mediated phenotypes in mice (Y. X. Wang et al., Circulation, 111, 2219 (2005)).

Since ERK1/2 is a pro: proliferative intracellular mediator, the reduction in aortic root growth achieved in Fbn1^{C1039G/+} mice could simply have been a result of decreased somatic growth of the whole animal. We therefore weighed all mice at the end of the 2 month trial, and found that RDEA119 therapy did not significantly affect the weight of either WT or Fbn1^{C1039G/+} mice (FIG. 25). This supports the conclusion that the reduction in growth achieved by RDEA119 in Fbn1^{C1039G/+} mice was specific to the aorta, and was not simply a manifestation of reduced somatic growth of the animal.

SP600125 was administered using a dosing regimen (30 mg/kg twice-daily by intraperitoneal injection) that was previously shown to cause clinically-relevant JNK antagonism in other murine models of disease (P. R. Eynott et al., B. J. Pharmacol. 140, 1373 (2003)).

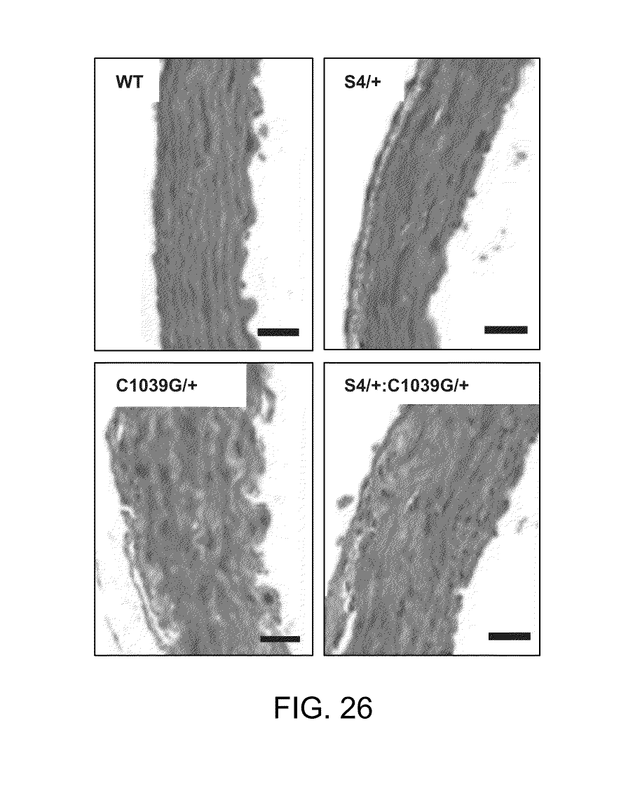

The exacerbation of aortic disease in Smad4 haploinsufficient MFS mice raises the question of whether Smad signaling is protective in MFS mice. For example, loss of Smad-driven collagen production in S4^{+/-}:Fbn1^{C1039G/+} mice could lead to aortic wall weakness and consequent rupture. Such a model is hard to support given the observation that both TGFβNAb and losartan achieve significant aortic protection in Fbn1^{C1039G/+} mice, despite their documented suppression of canonical TGFβ signaling. To further address this issue, we performed trichrome staining on the aortas of WT, S4^{+/-}, Fbn1^{C1039G/+} and S4^{+/-}:Fbn1^{C1039G/+} mice (FIG. 26). This shows a relative increase in collagen deposition in Fbn1^{C1039G/+} and S4^{+/-}:Fbn1^{C1039G/+} mice, compared to WT and S4^{+/-} littermates. It also shows that there is comparable aortic collagen content in Fbn1^{C1039G/+} and S4^{+/-}:Fbn1^{C1039G/+} mice, eliminating collagen deficiency as the mechanistic basis for aortic dissection and premature death in S4^{+/-}:Fbn1^{C1039G/+} mice.

Blockade of Smad activation (e.g. by Smad7 overexpression or by Smad2/3 siRNA) is an alternative approach to addressing the role of Smad signaling in MFS mice. However, these approaches would likely have resulted in increased inflammation, increased TGFβ ligand expression, and/or the activation of alternate pathways in our mice. Furthermore, Smad7 overexpressing mice die by 10 days of life (W. He et al., EMBO J. 21, 2580 (2002)), and in-vivo use of siRNA-based methods are extremely challenging. We therefore concluded that Smad4 haploinsufficiency was most likely the best way to reach meaningful conclusions. The fact that the clinical phenotype of Fbn1^{C1039G/+} and S4^{+/-}:Fbn1^{C1039G/+} mice showed invariant correlation with ERK and/or JNK signaling, but not Smad signaling, supports our conclusion that ERK and JNK are prominent drivers of aortic disease in MFS mice. Our data provide added incentive to explore new agents that inhibit ERK and/or JNK signaling. The long-term use of MAPK antagonists could theoretically have deleterious side effects. It is worth noting that both erk1−/− and erk1−/− erk2+/− mice survive, with no overt phenotypic defect. The mechanistic basis of the embryonic lethality seen in erk2−/− mice is not known, but it suggests that ERK2 is of critical importance during development. By contrast, inhibition of ERK1 and ERK2 activation by RDEA119 is well tolerated post-natally in mice, as well as in humans, and shows significant therapeutic benefit in our study. This appears to be analogous to TGFβ, where deficiency states are not tolerated during development, but are better tolerated and show phenotypic benefit postnatally. Finally, given losartan's profound inhibition of ERK activation, it is notable that losartan has been used for decades in patients without any apparent long-term deleterious consequences.

### INCORPORATION BY REFERENCE

The contents of all references, patents, pending patent applications and published patents, cited throughout this application are hereby expressly incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method of treating a subject having or at risk of developing a disease or disorder characterized by aberrant TGFβ expression or activity comprising:
administering to the subject an effective amount of an agent that modulates the activity of noncanonical TGFβ signaling; thereby treating the subject.

2. The method of claim 1, wherein the disease or disorder is Marfan syndrome or a clinical condition associated with Marfan syndrome.

3. The method of claim 2, wherein the disease or disorder is an aneurysm, an aortic aneurysm, or emphysema.

4. (canceled)

5. The method of claim 1, wherein the disease or disorder is a lung disease or disorder selected from the group consisting of emphysema, pneumothorax, and COPD.

6. (canceled)

7. The method of claim 1, wherein the agent is a noncanonical TGFβ signaling pathway inhibitor.

8. The method of claim 1, wherein the agent is an inhibitor of a molecule whose activity is required for ERK1/2 activation.

9. The method of claim 1, wherein the agent is an inhibitor of MEK, ERK1/2, or JNK1.

10. The method of claim 1, wherein the agent is an inhibitor of ERK1/2.

11. The method of claim 9, wherein the agent is selected from the group consisting of SP600125, U0126, and RDEA119.

12. The method of claim 1, wherein the agent is a siRNA or shRNA specific for a regulator of the noncanonical TGFβ signaling pathway.

13. The method of claim 12, wherein the siRNA or shRNA is specific for the nucleic acid molecule set forth as SEQ ID NO:1, SEQ ID NO:2, SEQ ID NO:3, SEQ ID NO:4, or SEQ ID NO:5.

14. A method of treating a subject having Marfan syndrome or a Marfan-associated condition comprising:
administering to the subject an effective amount of an agent that modulates the activity of noncanonical TGFβ signaling; thereby treating the subject.

15-19. (canceled)

20. A method of treating a subject having Marfan syndrome or a Marfan-associated condition comprising:
administering to the subject an effective amount of an agent that selectively activates Angiotensin II Receptor Type 2 (AT2); thereby treating the subject.

21. The method of claim 20, wherein the agent is a selective agonist of AT2.

22. (canceled)

23. A method of treating a subject having or at risk of developing a disease or disorder caused by mutation in the fibrillin 1 gene (Fbn1) comprising:
administering to the subject an effective amount of an agent that modulates the activity of noncanonical TGFβ signaling; thereby treating the subject.

24. The method of claim 23, wherein the disease or disorder is tissue fibrosis or scleroderma.

25-31. (canceled)

32. A pharmaceutical composition for the treatment of a disease or disorder characterized by aberrant TGFβ expression or activity, wherein the pharmaceutical composition comprises an agent that modulates the activity of noncanonical TGFβ signaling.

33. The pharmaceutical composition of claim 32, wherein the disease or disorder is Marfan syndrome or a clinical condition associated with Marfan syndrome, an aneurysm, an aortic aneurysm, emphysema, or a lung disease or disorder.

34-41. (canceled)

42. A kit for the treatment of a disease or disorder characterized by aberrant TGFβ expression or activity, wherein the pharmaceutical composition comprises an agent that modulates the activity of noncanonical TGFβ signaling, and instructions for use.

43. The kit of claim 42, wherein the disease or disorder is Marfan syndrome or a clinical condition associated with Marfan syndrome, an aneurism, an aortic aneurism, or emphysema a lung disease or disorder.

44-51. (canceled)

52. A method of optimizing the dosing regimen or route of delivery for a Marfan syndrome therapeutic comprising:
a) measuring noncanonical TGFβ signaling status in a sample from a subject;
b) increasing the dosage or altering the route of delivery of the Marfan syndrome therapeutic administered to the subject if the noncanonical TGFβ signaling is above a threshold amount; and
c) repeating steps a) and b) until the noncanoncial TGFβ signaling is below a threshold amount.

53. The method of claim 52, wherein the noncanonical TGFβ signaling status is MEK activity, ERK1/2 activity or JNK1 activity.


SEQUENCE LISTING (10 sequences)

SEQ ID NO: 1 - DNA, 1611 bp, Homo sapiens
acataatttctggagccctgtaccaacgtgtggccacatattctgtcaggaaccctgtgtgatcatggtctggatctgcaacacgggccaggccaaagtcacagatcttgagatcacaggtggtgttgagcagcaggcaggcaggcaatcggtccgagtggctgtcggctcttcagctctccgctcggcgtcttccttcctctcccggtcagcgtcggcggctgcaccggcggcgggcagtcctgcgggaggggcgacaagagctgaggcgcggccgccgagcgtcgagctcagcgcggcggaggcggcggcggcccggcagccaacatggcggcggcggcggcggcgggcgcgggcccggagatggtccgcgggcaggtgttcgacgtggggccgcgctacaccaacctctcgtacatcggcgagggcgcctacggcatggtgtgctctgcttatgataatgtcaacaaagttcgagtagctatcaagaaaatcagcccctttgagcaccagacctactgccagagaaccctgagggagataaaaatcttactgcgcttcagacatgagaacatcattggaatcaatgacattattcgagcaccaaccatcgagcaaatgaaagatgtatatatagtacaggacctcatggaaacagatctttacaagctcttgaagacacaacacctcagcaatgaccatatctgctattttctctaccagatcctcagagggttaaaatatatccattcagctaacgttctgcaccgtgacctcaagccttccaacctgctgctcaacaccacctgtgatctcaagatctgtgactttggcctggcccgtgttgcagatccagaccatgatcacacagggttcctgacagaatatgtggccacacgttggtacagggctccagaaattatgttgaattccaagggctacaccaagtccattgatatttggtctgtaggctgcattctggcagaaatgctttccaacaggcccatctttccagggaagcattatcttgaccagctgaatcacattttgggtattcttggatccccatcacaagaagacctgaattgtataataaatttaaaagctaggaactatttgctttctcttccacacaaaaataaggtgccatggaacaggctgttcccaaatgctgactccaaagctctggacttattggacaaaatgttgacattcaacccacacaagaggattgaagtagaacaggctctggcccacccatatctggagcagtattacgacccgagtgacgagcccatcgccgaagcaccattcaagttcgacatggaattggatgacttgcctaaggaaaagctaaaagaactaatttttgaagagactgctagattccagccaggatacagatcttaaatttgtcaggacaagggctcagaggactggacgtgctcagacatcggtgttcttcttcccagttcttgacccctggtcctgtctccagcccgtcttggcttatccactttgactcctttgagccgtttggaggggcggtttctggtagttgtggcttttatgctttcaaagaatttcttcagtccagagaattcactggcc

SEQ ID NO: 2 - DNA, 1866 bp, Homo sapiens
cgttcctcggcgccgccggggccccagagggcagcggcagcaacagcagcagcagcagcagcgggagtggagatggcggcggcggcggctcaggggggcgggggcggggagccccgtagaaccgagggggtcggcccgggggtcccgggggaggtggagatggtgaaggggcagccgttcgacgtgggcccgcgctacacgcagttgcagtacatcggcgagggcgcgtacggcatggtcagctcggcctatgaccacgtgcgcaagactcgcgtggccatcaagaagatcagccccttcgaacatcagacctactgccagcgcacgctccgggagatccagatcctgctgcgcttccgccatgagaatgtcatcggcatccgagacattctgcgggcgtccaccctggaagccatgagagatgtctacattgtgcaggacctgatggagactgacctgtacaagttgctgaaaagccagcagctgagcaatgaccatatctgctacttcctctaccagatcctgcggggcctcaagtacatccactccgccaacgtgctccaccgagatctaaagccctccaacctgctcagcaacaccacctgcgaccttaagatttgtgatttcggcctggcccggattgccgatcctgagcatgaccacaccggcttcctgacggagtatgtggctacgcgctggtaccgggccccagagatcatgctgaactccaagggctataccaagtccatcgacatctggtctgtgggctgcattctggctgagatgctctctaaccggcccatcttccctggcaagcactacctggatcagctcaaccacattctgggcatcctgggctccccatcccaggaggacctgaattgtatcatcaacatgaaggcccgaaactacctacagtctctgccctccaagaccaaggtggcttgggccaagcttttccccaagtcagactccaaagcccttgacctgctggaccggatgttaacctttaaccccaataaacggatcacagtggaggaagcgctggctcacccctacctggagcagtactatgacccgacggatgagccagtggccgaggagcccttcaccttcgccatggagctggatgacctacctaaggagcggctgaaggagctcatcttccaggagacagcacgcttccagcccggagtgctggaggccccctagcccagacagacatctctgcaccctggggcctggacctgcctcctgcctgcccctctcccgccagactgttagaaaatggacactgtgcccagcccggaccttggcagcccaggccggggtggagcatgggcctggccacctctctcctttgctgaggcctccagcttcaggcaggccaaggccttctcctccccacccgccctccccacggggcctcgggagctcaggtggccccagttcaatctcccgctgctgctgctgctgcgcccttaccttccccagcgtcccagtctctggcagttctggaatggaagggttctggctgccccaacctgctgaagggcagaggtggagggtggggggcgctgagtagggactcagggccatgcctgcccccctcatctcattcaaaccccaccctagtttccctgaaggaacattccttagtctcaagggctagcatccctgaggagccaggccgggccgaatcccctccctgtcaaagctgtcacttcgcgtgccctcgctgcttctgtgtgtggtgagcagaagtggagctggggggcgtggagagcccggcgcccctgccacctccctgacccgtctaatatataaatatagagatgtgtctatggctgaaaaaaaaaaaaaaaa

SEQ ID NO: 3 - DNA, 1418 bp, Homo sapiens
cattaattgcttgccatcatgagcagaagcaagcgtgacaacaatttttatagtgtagagattggagattctacattcacagtcctgaaacgatatcagaatttaaaacctataggctcaggagctcaaggaatagtatgcgcagcttatgatgccattcttgaaagaaatgttgcaatcaagaagctaagccgaccatttcagaatcagactcatgccaagcgggcctacagagagctagttcttatgaaatgtgttaatcacaaaaatataattggccttttgaatgttttcacaccacagaaatccctagaagaatttcaagatgtttacatagtcatggagctcatggatgcaaatctttgccaagtgattcagatggagctagatcatgaaagaatgtcctaccttctctatcagatgctgtgtggaatcaagcaccttcattctgctggaattattcatcgggacttaaagcccagtaatatagtagtaaaatctgattgcactttgaagattcttgacttcggtctggccaggactgcaggaacgagttttatgatgacgccttatgtagtgactcgctactacagagcacccgaggtcatccttggcatgggctacaaggaaaacgtggatttatggtctgtggggtgcattatgggagaaatggtttgccacaaaatcctctttccaggaagggactatattgatcagtggaataaagttattgaacagcttggaacaccatgtcctgaattcatgaagaaactgcaaccaacagtaaggacttacgttgaaaacagacctaaatatgctggatatagctttgagaaactcttccctgatgtccttttcccagctgactcagaacacaacaaacttaaagccagtcaggcaagggatttgttatccaaaatgctggtaatagatgcatctaaaaggatctctgtagatgaagctctccaacacccgtacatcaatgtctggtatgatccttctgaagcagaagctccaccaccaaagatccctgacaagcagttagatgaaagggaacacacaatagaagagtggaaagaattgatatataaggaagttatggacttggaggagagaaccaagaatggagttatacgggggcagccctctcctttagcacaggtgcagcagtgatcaatggctctcagcatccatcatcatcgtcgtctgtcaatgatgtgtcttcaatgtcaacagatccgactttggcctctgatacagacagcagtctagaagcagcagctgggcctctgggctgctgtagatgactacttgggccatcggggggtgggagggatggggagtcggttagtcattgatagaactactttgaaaacaattcagtggtcttatttttgggtgatttttcaaaaaatgta

SEQ ID NO: 4 - DNA, 2603 bp, Homo sapiens
aggcgaggcttccccttccccgcccctcccccggcctccagtccctcccagggccgcttcgcagagcggctaggagcacggcggcggcggcactttccccggcaggagctggagctgggctctggtgcgcgcgcggctgtgccgcccgagccggagggactggttggttgagagagagagaggaagggaatcccgggctgccgaaccgcacgttcagcccgctccgctcctgcagggcagcctttcggctctctgcgcgcgaagccgagtcccgggcgggtggggcgggggtccactgagaccgctaccggcccctcggcgctgacgggaccgcgcggggcgcacccgctgaaggcagccccggggcccgcggcccggacttggtcctgcgcagcgggcgcggggcagcgcagcgggaggaagcgagaggtgctgccctccccccggagttggaagcgcgttacccgggtccaaaatgcccaagaagaagccgacgcccatccagctgaacccggcccccgacggctctgcagttaacgggaccagctctgcggagaccaacttggaggccttgcagaagaagctggaggagctagagcttgatgagcagcagcgaaagcgccttgaggcctttcttacccagaagcagaaggtgggagaactgaaggatgacgactttgagaagatcagtgagctgggggctggcaatggcggtgtggtgttcaaggtctcccacaagccttctggcctggtcatggccagaaagctaattcatctggagatcaaacccgcaatccggaaccagatcataagggagctgcaggttctgcatgagtgcaactctccgtacatcgtgggcttctatggtgcgttctacagcgatggcgagatcagtatctgcatggagcacatggatggaggttctctggatcaagtcctgaagaaagctggaagaattcctgaacaaattttaggaaaagttagcattgctgtaataaaaggcctgacatatctgagggagaagcacaagatcatgcacagagatgtcaagccctccaacatcctagtcaactcccgtggggagatcaagctctgtgactttggggtcagcgggcagctcatcgactccatggccaactccttcgtgggcacaaggtcctacatgtcgccagaaagactccaggggactcattactctgtgcagtcagacatctggagcatgggactgtctctggtagagatggcggttgggaggtatcccatccctcctccagatgccaaggagctggagctgatgtttgggtgccaggtggaaggagatgcggctgagaccccacccaggccaaggacccccgggaggccccttagctcatacggaatggacagccgacctcccatggcaatttttgagttgttggattacatagtcaacgagcctcctccaaaactgcccagtggagtgttcagtctggaatttcaagattttgtgaataaatgcttaataaaaaaccccgcagagagagcagatttgaagcaactcatggttcatgcttttatcaagagatctgatgctgaggaagtggattttgcaggttggctctgctccaccatcggccttaaccagcccagcacaccaacccatgctgctggcgtctaagtgtttgggaagcaacaaagagcgagtcccctgcccggtggtttgccatgtcgcttttgggcctccttcccatgcctgtctctgttcagatgtgcatttcacctgtgacaaaggatgaagaacacagcatgtgccaagattctactcttgtcatttttaatattactgtctttattcttattactattattgttcccctaagtggattggctttgtgcttggggctatttgtgtgtatgctgatgatcaaaacctgtgccaggctgaattacagtgaaattttggtgaatgtgggtagtcattcttacaattgcactgctgttcctgctccatgactggctgtctgcctgtattttcgggattctttgacatttggtggtactttattcttgctgggcatactttctctctaggagggagccttgtgagatccttcacaggcagtgcatgtgaagcatgctttgctgctatgaaaatgagcatcagagagtgtacatcatgttattttattattattatttgcttttcatgtagaactcagcagttgacatccaaatctagccagagcccttcactgccatgatagctggggcttcaccagtctgtctactgtggtgatctgtagacttctggttgtatttctatatttattttcagtatactgtgtgggatacttagtggtatgtctctttaagttttgattaatgtttcttaaatggaattattttgaatgtcacaaattgatcaagatattaaaatgtcggatttatctttccccatatccaagtaccaatgctgttgtaaacaacgtgtatagtgcctaaaattgtatgaaaatccttttaaccattttaacctagatgtttaacaaatctaatctcttattctaataaatatactatgaaataaaaaaaaaaggatgaaagctaaaaaaaaaaaaaaaaaa

SEQ ID NO: 5 - DNA, 1759 bp, Homo sapiens
cccctgcctctcggactcgggctgcggcgtcagccttcttcgggcctcggcagcggtagcggctcgctcgcctcagccccagcgcccctcggctaccctcggcccaggcccgcagcgccgcccgccctcggccgccccgacgccggcctgggccgcggccgcagccccgggctcgcgtaggcgccgaccgctcccggcccgccccctatgggccccggctagaggcgccgccgccgccggcccgcggagccccgatgctggcccggaggaagccggtgctgccggcgctcaccatcaaccctaccatcgccgagggcccatcccctaccagcgagggcgcctccgaggcaaacctggtggacctgcagaagaagctggaggagctggaacttgacgagcagcagaagaagcggctggaagcctttctcacccagaaagccaaggtcggcgaactcaaagacgatgacttcgaaaggatctcagagctgggcgcgggcaacggcggggtggtcaccaaagtccagcacagaccctcgggcctcatcatggccaggaagctgatccaccttgagatcaagccggccatccggaaccagatcatccgcgagctgcaggtcctgcacgaatgcaactcgccgtacatcgtgggcttctacggggccttctacagtgacggggagatcagcatttgcatggaacacatggacggcggctccctggaccaggtgctgaaagaggccaagaggattcccgaggagatcctggggaaagtcagcatcgcggttctccggggcttggcgtacctccgagagaagcaccagatcatgcaccgagatgtgaagccctccaacatcctcgtgaactctagaggggagatcaagctgtgtgacttcggggtgagcggccagctcatcgactccatggccaactccttcgtgggcacgcgctcctacatggctccggagcggttgcagggcacacattactcggtgcagtcggacatctggagcatgggcctgtccctggtggagctggccgtcggaaggtaccccatccccccgcccgacgccaaagagctggaggccatctttggccggcccgtggtcgacggggaagaaggagagcctcacagcatctcgcctcggccgaggccccccgggcgccccgtcagcggtcacgggatggatagccggcctgccatggccatctttgaactcctggactatattgtgaacgagccacctcctaagctgcccaacggtgtgttcacccccgacttccaggagtttgtcaataaatgcctcatcaagaacccagcggagcgggcggacctgaagatgctcacaaaccacaccttcatcaagcggtccgaggtggaagaagtggattttgccggctggttgtgtaaaaccctgcggctgaaccagcccggcacacccacgcgcaccgccgtgtgacagtggccgggctccctgcgtcccgctggtgacctgcccaccgtccctgtccatgccccgcccttccagctgaggacaggctggcgcctccacccaccctcctgcctcacccctgcggagagcaccgtggcggggcgacagcgcatgcaggaacgggggtctcctctcctgcccgtcctggccggggtgcctctggggacgggcgacgctgctgtgtgtggtctcagaggctctgcttccttaggttacaaaacaaaacagggagagaaaaagcaaaaaaaaaaaaaaaaaaaaaaaaaaaa

SEQ ID NO: 6 - protein, 360 aa, Homo sapiens [sequence omitted]

SEQ ID NO: 7 - protein, 379 aa, Homo sapiens
MAAAAAQGGGGGEPRRTEGVGPGVPGEVEMVKGQPFDVGPRYTQLQYIGEGAYGMVSSAYDHVRKTRVAIKKISPFEHQTYCQRTLREIQILLRFRHENVIGIRDILRASTLEAMRDVYIVQDLMETDLYKLLKSQQLSNDHICYFLYQILRGLKYIHSANVLHRDLKPSNLLINTTCDLKICDFGLARIADPEHDHTGFLTEYVATRWYRAPEIMLNSKGYTKSIDIWSVGCILAEMLSNRPIFPGKHYLDQLNHILGILGSPSQEDLNCIINMKARNYLQSLPSKTKVAWAKLFPKSDSKALDLLDRMLTFNPNKRITVEEALAHPYLEQYYDPTDEPVAEEPFTFAMELDDLPKERLKELIFQETARFQPGVLEAP

SEQ ID NO: 8 - protein, 427 aa, Homo sapiens
MSRSKRDNNFYSVEIGDSTFTVLKRYQNLKPIGSGAQGIVCAAYDAILERNVAIKKLSRPFQNQTHAKRAYRELVLMKCVNHKNIIGLLNVFTPQKSLEEFQDVYIVMELMDANLCQVIQMELDHERMSYLLYQMLCGIKHLHSAGIIHRDLKPSNIVVKSDCTLKILDFGLARTAGTSFMMTPYVVTRYYRAPEVILGMGYKENVDLWSVGCIMGEMVCHKILFPGRDYIDQWNKVIEQLGTPCPEFMKKLQPTVRTYVENRPKYAGYSFEKLFPDVLFPADSEHNKLKASQARDLLSKMLVIDASKRISVDEALQHPYINVWYDPSEAEAPPPKIPDKQLDEREHTIEEWKELIYKEVMDLEERTKNGVIRGQPSPLGAAVINGSQHPSSSSSVNDVSSMSTDPTLASDTDSSLEAAAGPLGCCR

SEQ ID NO: 9 - protein, 393 aa, Homo sapiens [sequence omitted]

SEQ ID NO: 10 - protein, 400 aa, Homo sapiens [sequence omitted]